SEQUENCE LISTING (53 sequences)

SEQ ID NO: 1 - protein, 9 aa, synthetic construct
  REGION 1..9: note = Synthetic Peptide
LLFGVPVYV

SEQ ID NO: 2 - protein, 9 aa, synthetic construct
  REGION 1..9: note = Synthetic Peptide
SLLMWITQA

SEQ ID NO: 3 - protein, 10 aa, synthetic construct
  REGION 1..10: note = Synthetic Peptide
GLLGIGILTV

SEQ ID NO: 4 - protein, 9 aa, synthetic construct
  REGION 1..9: note = Synthetic Peptide
ETAGVPADV

SEQ ID NO: 5 - protein, 10 aa, synthetic construct
  REGION 1..10: note = Synthetic Peptide
ETAGIGILTV

SEQ ID NO: 6 - protein, 9 aa, synthetic construct
  REGION 1..9: note = Synthetic Peptide
GLLGVPLYV

SEQ ID NO: 7 - protein, 9 aa, synthetic construct
  REGION 1..9: note = Synthetic Peptide
YPNVNIHNF

SEQ ID NO: 8 - protein, 10 aa, synthetic construct
  REGION 1..10: note = Synthetic Peptide
RGPGRAFVFI

SEQ ID NO: 9 - protein, 10 aa, synthetic construct
  REGION 1..10: note = Synthetic Peptide
EAAGIGILTV

SEQ ID NO: 10 - protein, 9 aa, synthetic construct
  REGION 1..9: note = Synthetic Protein
VYGFVRACL

SEQ ID NO: 11 - protein, 10 aa, synthetic construct
  REGION 1..10: note = Synthetic Protein
RYPLTFGWCF

SEQ ID NO: 12 - protein, 9 aa, synthetic construct
  REGION 1..9: note = Synthetic Protein
ALNTLVKQL

SEQ ID NO: 13 - protein, 9 aa, synthetic construct
  REGION 1..9: note = Synthetic Peptide
VLNDILSRL

SEQ ID NO: 14 - protein, 9 aa, synthetic construct
  REGION 1..9: note = Synthetic Peptide
RLNEVAKNL

SEQ ID NO: 15 - protein, 9 aa, synthetic construct
  REGION 1..9: note = Synthetic Peptide
NLNESLIDL

SEQ ID NO: 16 - protein, 9 aa, synthetic construct
  REGION 1..9: note = Synthetic Peptide
FIAGLIAIV

SEQ ID NO: 17 - protein, 9 aa, synthetic construct
  REGION 1..9: note = Synthetic Peptide
GLMWLSYFI

SEQ ID NO: 18 - protein, 9 aa, synthetic construct
  REGION 1..9: note = Synthetic Peptide
LLLDRLNQL

SEQ ID NO: 19 - protein, 9 aa, synthetic construct
  REGION 1..9: note = Synthetic Peptide
GMSRIGMEV

SEQ ID NO: 20 - protein, 9 aa, synthetic construct
  REGION 1..9: note = Synthetic Peptide
WLMWLIINL

SEQ ID NO: 21 - protein, 9 aa, synthetic construct
  REGION 1..9: note = Synthetic Peptide
ILLLDQALV

SEQ ID NO: 22 - protein, 9 aa, synthetic construct
  REGION 1..9: note = Synthetic Peptide
SLPGVFCGV

SEQ ID NO: 23 - protein, 10 aa, synthetic construct
  REGION 1..10: note = Synthetic Peptide
TLACFVLAAV

SEQ ID NO: 24 - protein, 10 aa, synthetic construct
  REGION 1..10: note = Synthetic Peptide
KLPDDFTGCV

SEQ ID NO: 25 - protein, 99 aa, synthetic construct [sequence omitted]

SEQ ID NO: 26 - protein, 277 aa, synthetic construct
  REGION 1..277: note = Synthetic HLA-A*02:01
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPS

SEQ ID NO: 27 - protein, 278 aa, synthetic construct
  REGION 1..278: note = Synthetic HLA-A*24:02
GSSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPS

SEQ ID NO: 28 - protein, 277 aa, synthetic construct
  REGION 1..277: note = Synthetic HLA-A*68:02
GSHSMRYFYTSMSRPGRGEPRFIAVGYVDDTQFVRFDSDAASQRMEPRAPWIEQEGPEYWDRNTRNVKAQSQTDRVDLGTLRGYYNQSEAGSHTIQRMYGCDVGPDGRFLRGYHQYAYDGKDYIALKEDLRSWTAADMAAQTTKHKWEAAHVAEQWRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLKWEPS

SEQ ID NO: 29 - protein, 48 aa, synthetic construct
  REGION 1..48: note = Synthetic Peptide
EVDGGGGGLTDTLQAETDQLEDEKSALQTEIANLLKEKEKLEFILAAH

SEQ ID NO: 30 - protein, 7 aa, synthetic construct
  REGION 1..7: note = Synthetic Peptide
ENLYFQS

SEQ ID NO: 31 - protein, 16 aa, synthetic construct
  REGION 1..16: note = Synthetic Peptide
GGGSGGGSGGGSGGGS

SEQ ID NO: 32 - protein, 4 aa, synthetic construct
  REGION 1..4: note = Synthetic Peptide
SQSG

SEQ ID NO: 33 - protein, 5 aa, synthetic construct
  REGION 1..5: note = Synthetic Peptide
GGGGS

SEQ ID NO: 34 - protein, 15 aa, synthetic construct
  REGION 1..15: note = Synthetic Peptide
GLNDIFEAQKIEWHE

SEQ ID NO: 35 - protein, 496 aa, synthetic construct
  REGION 1..496: note = Synthetic Peptide
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGSGGGSGGGSGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQSGGLNDIFEAQKIEWHEGGGGSENLYFQSGGGGSEVDGGGGGLTDTLQAETDQLEDEKSALQTEIANLLKEKEKLEFILAAH

SEQ ID NO: 36 - protein, 501 aa, synthetic construct
  REGION 1..501: note = Synthetic Peptide
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQSGGLNDIFEAQKIEWHEGGGGSENLYFQSGGGGSEVDGGGGGLTDTLQAETDQLEDEKSALQTEIANLLKEKEKLEFILAAH

SEQ ID NO: 37 - protein, 500 aa, synthetic construct
  REGION 1..500: note = Synthetic Peptide
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSMSRPGRGEPRFIAVGYVDDTQFVRFDSDAASQRMEPRAPWIEQEGPEYWDRNTRNVKAQSQTDRVDLGTLRGYYNQSEAGSHTIQRMYGCDVGPDGRFLRGYHQYAYDGKDYIALKEDLRSWTAADMAAQTTKHKWEAAHVAEQWRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLKWEPSSQSGGLNDIFEAQKIEWHEGGGGSENLYFQSGGGGSEVDGGGGGLTDTLQAETDQLEDEKSALQTEIANLLKEKEKLEFILAAH

SEQ ID NO: 38 - protein, 387 aa, synthetic construct
  REGION 1..387: note = Synthetic Peptide
KPHPAEGQWRAVDVVLDCFLVKDGAHRGALASSEDRARASLVLKQVPVLDDGSLEDFTDFQGGTLAQDDPPIIFEASVDLVQIPQAEALLHADASGKEVTCEISRYFLQMTETTVKTAAWFMANVQVSGGGPSISLVMKTPRVAKNEVLWHPTLNLPLSPQGTVRTAVEFQVMTQTQSLSFLLGSSASLDCGFSMAPGLDLISVEWRLQHKGRGQLVYSWTAGQGQAVRKGATLEPAQLGMARDASLTLPGLTIQDEGTYICQITTSLYRAQQIIQLNIQASPKVRLSLANEALLPTLICDIAGYYPLDVVVTWTREELGGSPAQVSGASFSSLRQSVAGTYSISSSLTAEGSAGATYTCQVTHISLEEPLGASTQVVPPERRLEGA

SEQ ID NO: 39 - protein, 46 aa, synthetic construct
  REGION 1..46: note = Synthetic Peptide
KVDGGGGGRIARLEEKVKTLKAQNSELASTANMLREQVAQLKQVMN

SEQ ID NO: 40 - protein, 18 aa, synthetic construct
  REGION 1..18: note = Synthetic Peptide
GGSGGGGSGGGASGGGGS

SEQ ID NO: 41 - protein, 6 aa, synthetic construct
  REGION 1..6: note = Synthetic Peptide
SGGGGS

SEQ ID NO: 42 - protein, 11 aa, synthetic construct
  REGION 1..11: note = Synthetic Peptide
GGGSGGGSGGS

SEQ ID NO: 43 - protein, 8 aa, synthetic construct
  REGION 1..8: note = Synthetic Peptide
WSHPQFEK

SEQ ID NO: 44 - protein, 494 aa, synthetic construct
  REGION 1..494: note = Synthetic Peptide
KPHPAEGQWRAVDVVLDCFLVKDGAHRGALASSEDRARASLVLKQVPVLDDGSLEDFTDFQGGTLAQDDPPIIFEASVDLVQIPQAEALLHADASGKEVTCEISRYFLQMTETTVKTAAWFMANVQVSGGGPSISLVMKTPRVAKNEVLWHPTLNLPLSPQGTVRTAVEFQVMTQTQSLSFLLGSSASLDCGFSMAPGLDLISVEWRLQHKGRGQLVYSWTAGQGQAVRKGATLEPAQLGMARDASLTLPGLTIQDEGTYICQITTSLYRAQQIIQLNIQASPKVRLSLANEALLPTLICDIAGYYPLDVVVTWTREELGGSPAQVSGASFSSLRQSVAGTYSISSSLTAEGSAGATYTCQVTHISLEEPLGASTQVVPPERRLEGAGGSGGGGSGGGASGGGGSENLYFQSSGGGGSKVDGGGGGRIARLEEKVKTLKAQNSELASTANMLREQVAQLKQVMNWSHPQFEKGGGSGGGSGGSAWSHPQFEKAA

SEQ ID NO: 45 - protein, 9 aa, synthetic construct
  REGION 1..9: note = Synthetic Peptide
NLVPMVATV

SEQ ID NO: 46 - protein, 4 aa, synthetic construct
  REGION 1..4: note = Synthetic Peptide linker
GGGS

SEQ ID NO: 47 - protein, 16 aa, synthetic construct
  REGION 1..16: note = Synthetic Peptide linker
GGGSGGGSGGGSGGGS

SEQ ID NO: 48 - protein, 4 aa, synthetic construct
  REGION 1..4: note = Synthetic Peptide
DDDK

SEQ ID NO: 49 - protein, 6 aa, synthetic construct
  REGION 1..6: note = Synthetic Peptide
LVPRGS

SEQ ID NO: 50 - protein, 8 aa, synthetic construct
  REGION 1..8: note = Synthetic Peptide
LEVLFQGP

SEQ ID NO: 51 - protein, 15 aa, synthetic construct
  REGION 1..15: note = Synthetic Peptide
GYYNQSEAGSHTVQR

SEQ ID NO: 52-53 - skipped (2 entries)

(19) United States
(12) Patent Application Publication
(10) Pub. No.: US 2024/0262885 A1
(43) Pub. Date: Aug. 8, 2024

(54) SYSTEMS AND METHODS FOR THE PREPARATION OF PEPTIDE-MHC-I COMPLEXES WITH NATIVE GLYCAN MODIFICATIONS

(71) Applicant: The Regents of The University of California, Oakland, CA (US)

(72) Inventors: Nikolaos G. Sgourakis, Philadelphia, PA (US); Sara O'Rourke, Scotts Valley, CA (US)

(21) Appl. No.: 18/528,645

(22) Filed: Dec. 4, 2023

Related U.S. Application Data
(63) Continuation of application No. 17/017,685, filed on Sep. 11, 2020, now abandoned.
(60) Provisional application No. 62/900,260, filed on Sep. 13, 2019.
(60) Provisional application No. 63/047,812, filed on Jul. 2, 2020.
(60) Provisional application No. 63/011,221, filed on Apr. 16, 2020.
(60) Provisional application No. 63/076,601, filed on Sep. 10, 2020.
(60) Provisional application No. 62/957,040, filed on Jan. 3, 2020.

(51) Int. Cl. C07K 14/74

(52) U.S. Cl. CPC C07K 14/70539; C07K 2317/51; C07K 2319/10; C07K 2319/22

(57) ABSTRACT

Disclosed herein are novel glycosylated peptide receptive MHC-I complexes that allow for efficient production of glycosylated MHC-I multimers. Such glycosylated peptide receptive MHC-I complexes include a single-chain MHC-I construct and are produced in mammalian expression systems (e.g., CHO and HEK cells) that allow for the glycosylation of the complexes at one or more native positions. Multimers (e.g., tetramers) produced from the glycosylated peptide receptive MHC-I complexes provided herein advantageously allow for the identification of high-affinity T cell and natural killer cell receptors previously unidentified using traditional unglycosylated MHC tetramers.

### CROSS REFERENCE TO RELATED APPLICATION

This application is a Continuation of U.S. application Ser. No. 17/017,685 filed Sep. 11, 2020, which claims priority to U.S. Provisional Patent Application No. 62/900,260, filed Sep. 13, 2019, U.S. Provisional Patent Application No. 63/047,812, filed Jul. 2, 2020, U.S. Provisional Patent Application No. 63/011,221, filed on Apr. 16, 2020, U.S. Provisional Patent Application No. 63/076,601, filed on Sep. 10, 2020, and U.S. Provisional Patent Application No. 62/957,040, filed on Jan. 3, 2020, which are hereby incorporated by reference in their entirety.

### REFERENCE TO SEQUENCE LISTING SUBMITTED ELECTRONICALLY

This application contains a Sequence Listing which has been submitted electronically in XML file format and is hereby incorporated by reference in its entirety. The XML file, named 116550-01-5011-US01.xml, was created on Apr. 27, 2024 and is 67 kilobytes in size.

### BACKGROUND

The class I molecules of the Major Histocompatibility Complex (MHC) play a pivotal role in orchestrating an adaptive immune response by alerting the immune system to the presence of developing infections and tumors in the body. Immune surveillance is achieved through the display of short (8-11 residue long) peptides derived from viral proteins or mutated oncogenes via a tight interaction with the MHC-I peptide-binding groove. Such peptide/MHC-I protein complexes are assembled inside the cell and displayed on the surface of all antigen-presenting cells where they can interact with specialized receptors on T cells and natural killer (NK) cells. The MHC-I proteins are extremely polymorphic (more than 13,000 different alleles have been identified in the human population to date), and each allele can display an estimated 1,000-10,000 different peptides, which makes the characterization of specific T cell responses against a panel of known peptide epitopes a daunting task. Further adding to the challenge of characterizing such T cell responses is the fact that typical T cell receptor affinities for their cross-reactive pMHC (peptide loaded MHC) ligands are low (in the micromolar range).

Multivalent, fluorescent pMHC-I multimers (Altman J D et al., Science 274, 94-96 (1996); incorporated by reference herein) were developed and used to stain T cells (Altman and Davis, Curr Protoc Immunol Ch. 17 Unit 17.3 (2003); incorporated by reference herein). T cells that recognize a specific peptide/MHC multimer can be identified and sorted using flow cytometry, and their receptors can be identified in subsequent steps. Peptide loaded MHC-I tetramers have revolutionized experimental immunology and the development of new therapies, leading to a breadth of discoveries (Doherty, J Immunol 187, 5-6 (2011); incorporated by reference herein). However, the preparation of properly conformed pMHC molecules via in vitro refolding of inclusion bodies expressed in E. coli (Garboczi D N et al., PNAS 89, 3429-3433 (1992); incorporated by reference herein), requires a laborious, multi-step process that is highly inefficient (typical refolding yields are <5% by weight).

Moreover, all MHC molecules expressed in E. coli lack the functionally relevant post-translational glycosylation that is required for proper immune surveillance function (Barber L D et al., J Immunol 156, 3275-3284 (1996); incorporated by reference herein). For example, in E. coli expressed MHC molecules, a conserved glycan at residue N86, which is present in all human HLA-A, HLA-B, and HLA-C alleles, and is located at a site near the TCR recognition surface, is completely missing. Therefore, E. coli expressed refolded tetramers will fail to identify high-affinity T cell receptors and natural killer cell receptors that have binding to an MHC molecule, where that binding is dependent on glycosylation. Such TCRs can be important targets for both understanding antigen recognition processes, and the development of immunotherapies to combat bacterial and viral infections and cancer.

Thus, there remains a need for novel MHC-I tetramer compositions that allow for the identification of such previously unidentified T cell and NK receptors, as well as methods for making such tetramers in an efficient manner.

### SUMMARY

Disclosed herein are novel glycosylated peptide receptive MHC-I complexes that allow for efficient production of pMHC-I multimers that can be used, for example, as T cell/NK cell staining reagents and drug delivery vehicles. Such glycosylated peptide receptive MHC-I complexes include an MHC-I (e.g., a single-chain MHC-I) and are produced in mammalian expression systems (e.g., CHO and HEK cells) that allow for the glycosylation of the complexes at one or more native amino acid positions (e.g., at the conserved N86 in HLA-A, HLA-B, and HLA-C). The protein constructs used to make such glycosylated peptide receptive MHC-I complexes include leucine zipper domains and one or more purification tags that facilitate purification of the complexes. The subject peptide receptive MHC-I/TAPBPR complexes are capable of binding high-affinity peptides with the correct peptide specificity (FIGS. 7 and 8). Further, the glycosylated peptide receptive MHC-I complexes can be used to produce pMHC multimer libraries for basic research, diagnostic and therapeutic applications. Such multimers (e.g., tetramers) produced from the glycosylated peptide receptive MHC-I complexes provided herein advantageously allow for the identification of high-affinity T cell and natural killer cell receptors previously unidentified using traditional unglycosylated MHC tetramers.

In a first aspect, provided herein are MHC-I protein constructs. In some embodiments, the MHC-I protein constructs include: (a) a first polypeptide that includes a MHC Class I heavy chain that is glycosylated at least one native glycosylation position; and (b) a second polypeptide that includes a β2 microglobulin. In some embodiments, the constructs further include: (c) a third polypeptide that includes a leucine zipper domain; and (d) a fourth polypeptide that includes a protease cleavage site. In exemplary embodiments, (a), (b), (c), and (d), are covalently linked from N- to C-terminus orientation according to the following order: (b)-(a)-(d)-(c).

In some embodiments, the β2 microglobulin is N-terminal to the MHC Class I heavy chain, and the MHC-I protein construct further includes a first peptide linker between the β2 microglobulin and the MHC Class I heavy chain.

In some embodiments, the leucine zipper domain is C-terminal to the MHC Class I heavy chain, and the protease cleavage site is between the leucine zipper domain and the MHC Class I heavy chain. In certain embodiments, the MHC-I protein construct further includes a second peptide linker between the MHC Class I heavy chain and the protease cleavage site and a third peptide linker between the protease cleavage site and the leucine zipper domain.

In exemplary embodiments, the MHC-I protein construct further includes a (e) multimerization tag (e.g., an AviTag). In some ebodiments, the multimerization tag is C-terminal to the MHC Class I heavy chain. In certain embodiments, the multimerization tag is C-terminal to the MHC Class I heavy chain and N-terminal to the protease cleavage site such that the tag remains bound to the MHC Class I heavy chain after protease cleavage. In further embodiments, the MHC-I protein construct further includes a fourth peptide linker between the MHC Class I heavy chain and the multimerization tag.

In some embodiments, the MHC-I protein constructs include: (a) a first polypeptide that includes a MHC Class I heavy chain, (b) a second polypeptide that includes a β2 microglobulin; (c) a third polypeptide that includes a leucine zipper domain; (d) a fourth polypeptide that includes a protease cleavage site; and (e) a multimerization tag, where (a), (b), (c), (d) and (e) are covalently linked from N- to C-terminus orientation according to the following order: (b)-(a)-(e)-(d)-(c). In an exemplary, the MHC-I protein constructs further include (f) one or more purification tags. In some embodiments that include the (f) one or more purification tags, (a), (b), (c), (d) and (f) are covalently linked from N-to C-terminus orientation according to the following order: (b)-(a)-(d)-(c)-(f).

In some embodiments, the MHC Class I heavy chain of the MHC-I protein construct is a human HLA-A, HLA-B, or HLA-C or a mouse H-2D or H-2L. In exemplary embodiments, the MHC Class I heavy chain is an HLA-A*02:01, HLA-A*24:02, HLA-A*68:01 or HLA-A*68:02 allele heavy chain. In certain embodiments, MHC Class I heavy chain has one or more mutations in the α3 domain of the heavy chain.

In exemplary embodiments, the protease cleavage site is a TEV protease cleavage site. In some embodiments, the multimerization tag of the MHC protein construct is an AviTag. In particular embodiments, the AviTag includes a biotinylated lysine.

In exemplary embodiments of the MHC-I protein construct, the MHC class I heavy chain is glycosylated at residue N86.

In another aspect, provided herein is a TAPBPR protein construct that includes: (a) a first polypeptide that includes a TAPBPR; (b) a second polypeptide that includes a leucine zipper domain; and (c) a third polypeptide that includes a protease cleavage site. In exemplary embodiments, (a), (b), and (c) are covalently linked from N-to C-terminus orientation according to the following order: (a)-(c)-(b).

In some embodiments, the leucine zipper domain is C-terminal to the TAPBPR and the protease cleavage site is between the TAPBPR and the leucine zipper domain. In certain embodiments, the TAPBPR protein construct includes a first peptide linker between the TAPBPR and the protease cleavage site and a second peptide linker between the protease cleavage site and the leucine zipper domain.

In some embodiments, the TAPBPR protein construct further includes:(d) one or more purification tags. In certain embodiments, the purification are C-terminal to the leucine zipper domain such that the purification tag remains bound to the leucine zipper domain after protease cleavage.

In exemplary embodiments, the TAPBPR protein construct includes: (a) a first polypeptide that includes a TAPBPR; (b) a second polypeptide that includes a leucine zipper domain; and (c) a third polypeptide that includes a protease cleavage site; and (d) one or more purification tag , wherein (a), (b), (c), and (d) are covalently linked from N- to C-terminus orientation according to the following order: (a)-(c)-(b)-(d).

In some embodiments, the protease cleavage site is specific for a TEV protease.

In certain embodiments, the one or more purification tags include a first Strep-tag II® tag. In certain embodiments, the one or more purification tags also includes a second Strep-tag II® tag C-terminal to the first Strep-tag II® tag and a third peptide linker between the first Strep-tag II® tag and the second Strep-tag II® tag.

In another aspect, provided herein are polynucleotides encoding the MHC-I protein constructs and TAPBPR protein constructs provided herein.

In one aspect, provided herein are expression vectors that include polynucleotides encoding the MHC-I protein constructs and/or TAPBPR protein constructs provided herein. In some embodiments, the polynucleotide expression vector includes a first polynucleotide that encodes any one of the subject MHC-I protein constructs described herein and a second polynucleotide that encodes any one of the TAPBPR protein construct described herein. In particular embodiments, the expression vector further includes a CMV promoter.

In some embodiments, the expression vector is an expression vector composition that includes two expression vectors. The first expression vector includes a first polynucleotide that encodes any one of the subject MHC-I protein constructs described herein. The second expression vector includes a second polynucleotide that encodes any one of the TAPBPR protein construct described herein. In certain embodiments, the first and second polynucleotide expression vector each includes a CMV promoter.

In another aspect, provided herein are mammalian host cells that include any of the expression vectors provided herein. In certain embodiments, the host cell is a CHO or HEK cell. In particular embodiments, the host cell is a CHO-K1 cell.

In another aspect, provided herein are methods of making peptide receptive MHC-I complexes. In some embodiments, the method includes the steps of: a) providing a mammalian host cell that includes: i) a first polynucleotide that encodes for one of the MHC protein constructs provided herein, and ii) a second polynucleotide that encodes for one of the TAPBPR constructs provided herein, where the leucine zipper domain of the first protein construct specifically binds the leucine zipper domain of the second protein construct and where the protease cleavage site in the first protein construct is the same protease cleavage site as in the second protein construct; b) culturing the mammalian host cell in a culture medium under conditions where the MHC protein construct and TAPBPR construct are expressed; c) collecting the culture medium after culturing; d) applying the culture medium to a column that includes an agent that binds the first protein construct or second protein construct, thereby forming a zippered MHC-I/TAPBPR complex bound to the column, wherein the MHC-I/TAPBPR complex includes an MHC-I heavy chain that is glycosylated at least one native glycosylation position; e) eluting the zippered MHC-I/TAPBPR complex from the column; and f) contacting the zippered MHC-I/TAPBPR complex with a protease specific for the protease cleavage site of the first protein construct and the protease cleavage site of the second protein construct, thereby creating a purified peptide-receptive MHC-I complex. In some embodiments, the peptide receptive MHC-I complexes further comprise a TAPBPR protein.

In some embodiments, the mammalian host cell includes an expression vector or expression vector composition provided herein. In certain embodiments, the column includes streptavidin or Strep-tactin®. In some embodiments, the protease is TEV.

In exemplary embodiments, the leucine zipper domain of the first protein construct is Fos and the leucine zipper domain of the second protein construct is Jun. In other embodiments, the leucine zipper domain of the first protein construct is Jun and the leucine zipper domain of the second protein construct is Fos.

In another aspect, provided herein is a method of making a purified peptide receptive MHC-I complex. This method includes the steps of: a) providing a mammalian host cell that includes: i) a first polynucleotide that encodes for one of the MHC protein constructs provided herein, and ii) a second polynucleotide that encodes for a TAPBPR; b) culturing the mammalian host cell in a culture medium under conditions where the MHC protein construct and TAPBPR are co-expressed; and c) collecting the protein construct and TAPBPR.

In another aspect, provided herein is a method of making a tetrameric peptide MHC-I complex. The method includes the steps of: (a) contacting a plurality of purified peptide receptive MHC-I complexes with streptavidin, where the purified peptide receptive complexes include at least one biotinylated residue and an MHC-I heavy chain that is glycosylated at least one native glycosylation position, thereby making a tetrameric peptide receptive MHC-I complex; and (b) contacting the tetrameric peptide receptive MHC-I complex with a plurality of peptides of interest, thereby forming the tetrameric peptide-MHC-I complex. In some embodiments, the purified peptide receptive MHC-I complexes each include exactly one biotinylated residue.

In exemplary embodiments, the purified peptide receptive MHC-I complexes each include an AviTag that includes one lysine residue. In some embodiments, the method further includes the step of biotinylating the lysine residue in the AviTag. In certain embodiments, biotinylating the lysine residue in the AviTag includes contacting the purified peptide receptive MHC-I complexes with biotin in the presence of a biotin ligase enzyme (e.g., BirA). In particular embodiments, the streptavidin includes a fluorescent tag. In certain embodiments, at least one of the peptide receptive MHC-I complexes of the plurality of peptide receptive MHC-I complexes comprises a TAPBPR.

In another aspect, provided herein are tetrameric peptide-MHC class I complexes that include: a) a tetrameric streptavidin molecule comprised of four streptavidin subunits; and b) four peptide-MHC Class I complexes, where at least one of the pMHC-I is glycosylated at at least one native glycosylation position, and where each streptavidin subunit is bound via its biotin binding site to one of the four peptide-MHC Class I complexes.

In some embodiments, each of the peptide-MHC Class I complexes is glycosylated at residue N86 of the MHC Class I heavy chain. In certain embodiments, each of the four peptide-MHC Class I complexes includes a single-chain MHC-I, wherein the single-chain MHC-I comprises a MCH-I heavy chain covalently linked to a β2 microglobulin. In certain embodiments, the peptide-MHC Class I complexes further comprises a fluorescent tag. In particular embodiments, the fluorescent tag is attached to the tetrameric streptavidin molecule.

### BRIEF DESCRIPTION OF THE DRAWINGS

FIG. 1 provides a schematic for the production of glycosylated MHC-Class I tetramers using the glycosylated peptide receptive MHC-I complexes described herein and use of such glycosylated MHC-Class I tetramers for high-throughput T cell repertoire analysis. (A) Chinese Hamster (CHO) cells were co-transfected with a pair of plasmids expressing a single-chain MHC-I and the chaperone TAPBPR, with high affinity heterodimeric leucine zippers. Even in the absence of a high affinity peptide, a stable leucine zippered single-chain MHC-I/TAPBPR complex was over-expressed and secreted from the CHO cells. (B) Step-tag® affinity purification from culture supernatant is followed by removal of the leucine zippers by Tobacco Etch Virus (TEV) protease digestion. (C) The TAPBPR acts upon the MHC-I to create a glycosylated peptide receptive MHC-I complex. (D) The glycosylated peptide receptive MHC-I complexes may be biotinylated with biotin ligase, then tetramerized with streptavidin PE before storage or direct loading with high-affinity peptide. As illustrated here and without being bound by theory, high-affinity glycosylated peptide-MHC complex (pMHC) formation results from contacting peptides of interest with glycosylated peptide receptive MHC-I complexes, and the pMHC is ready for a range of research, diagnostic and therapeutic applications, including, for example, T cell repertoire analysis, receptor ligand characterization and T cell stimulation.

FIG. 2 shows the organization of an example of a single-chain MHC-I construct and an example of a TAPBPR chaperone protein construct. CMV expression cassettes were organized as shown. The single-chain MHC-I construct shown here comprises a beta2 microglobulin gene including endogenous secretory peptide, a 16 amino acid spacer, the HLA*A2:01 ectodomain, an AviTag (GLNDIFEAQKIEWHE) (SEQ ID NO:34) for biotinylation with BirA, a TEV protease site and a Fos zipper. The TAPBPR (luminal domain) gene was engineered to remove the unpaired cysteine at position 94 (Neerincx et al, eLife 6, e23049 (2017); incorporated by reference herein), and add a TEV cleavage site upstream from the Jun leucine zipper and Strep-tag® motifs (WSHPQFEK)(SEQ ID NO:43).

FIG. 3 provides another schematic showing the features of the single-chain MHC-I and TAPBPR chaperone protein constructs provided herein. Single-chain-Fos (Z1) and TAPBPR-Jun (Z2) constructs were ligated via HindIII and XBa1 enzyme restriction sites into an expression vector previously described by O'Rourke et al., PLOS One 13, e0197656 (2018); incorporated by reference herein. Flexible GS linkers were employed to covalently link the light and heavy chain MHC-I components, and essential elements of the recombinant molecule. A single AviTag located on the carboxy terminal of the mature MHC-I molecule, permits site specific biotinylation of a lysine (K) residue. The Strep-Tactin® peptide tags on the carboxy-terminal of the TAPBPR molecule permitted purification of zippered complex. Leucine zippers were then removed by digestion with TEV protease and “unzipped” complex isolated by size exclusion chromatography.

FIG. 4 provides a simplified view of the N-linked glycosylation pathway. N-linked glycosylation begins in the endoplasmic reticulum with the en-block transfer of a highly conserved Gluc₃Man₉GlcNac₂ structure (left) to asparagine residues within the N-X-S/T motif of nascent proteins. This initial structure is sequentially trimmed down to the α3 Gluc₁Man₉GlcNac₂ where the quality control calnexin/calreticulin either sends glycoproteins to be degraded or secreted as Man₉GlcNac₂ which is further trimmed to Man₅GlucNac₂ (center). Various glycosyltransferases then add monosaccharides creating hybrid (second from right) and complex (right) glycoforms. Kifunensine and Swainsonine are both inhibitors that halt further processing at the points shown above. EndoH and PNGase F remove the glycan structures where indicated by the arrows, with hybrid and complex glycans being insensitive to Endo H.

FIGS. 5A and 5B provide a summary of a study, showing expression of MHC-I/TAPBPR in transiently transfected CHOK1s C1s−/− cells. Twenty-four hours post transfection, cells were shifted to 32° C. culture and 1 mM sodium butyrate added. Cultures were fed daily with CHO Feed A and yeastolate and harvested at day 5. (A) and (B): Western Blot of 10 μl of conditioned media (harvested at day 3), (A) with an anti-TAPBPR polyclonal antibody or (B) monoclonal anti-B2M. 1 Std, 2-4 supernatant single-chain MHC I, supernatant TAPBPR, and supernatant MHC-I/TAPBPR respectively 5, single-chain MHC I/TAPBPR plus DTT; 6 supernatant no EP; 7-10 cell lysate single-chain MHC I, TAPBPR, single-chain MHC I/TAPBPR, single-chain MHC I/TAPBPR plus DTT respectively; 12 TAPBPR 47 kDa marker. Polyclonal TAPBPR has some cross reactivity with the MHC single-chain (lane 2) but the monoclonal antiB2M has no TAPBPR binding.

FIGS. 6A through 6D depict the purification of an exemplary glycosylated MHC-I/TAPBPR zippered complex. (A) capture of a streptactin-tag on the carboxy-terminal TAPBPR molecule efficiently isolates MHC-I/TAPBPR zippered complex which resolves on a 12% SDS PAGE gel as two bands at approximately 56 and 51 kDa. (Coomassie stained gel). TEV cleavage results in a reduction in size to 51 and 43.5 kDa respectively and the zippers between 8-14 kDa (which appear to heterodimerize in A). (B) Leucine zippered and “unzippered “complexes (36 μM) were resolved on an S200 10/30 column in 50 mM Tris pH 7.5 buffer containing 100 mM NaCl. The zippered MHC-I/TAPBPR complex peaks at 24 minutes (black line) and unzippered (TEV digested) complex peaks at 26.5-27 minutes (grey line). (C) Free TAPBPR and single-chain MHC-I molecules cannot be efficiently resolved on the S200 column, but fractions corresponding to the approx. 95 kDa unzippered MHC-I/TAPBPR complex (25-30 minutes) were pooled and concentrated. (D) Aliquots of protein eluted from the column were electrophoresed on a 12% SDS PAGE gel, confirming the presence of both single-chain MHC-I and TAPBPR in the elution fractions corresponding to the complex. Free TEV enzyme and the cleaved leucine zippers elute at larger volumes and can be observed as separate fractions.

FIGS. 7A and 7B depict a native gel shift assay of peptide binding to leucine zippered glycosylated MHC-I/TAPBPR complex. (A) “Zippered” MHC-I/TAPBPR was incubated with increasing molar ratios of TAX peptide, which has a high affinity for HLA-A*02:01, or a non-binding P18-I10 (P18) peptide, for 1 hour at room temperature in Tris buffer pH 7.5, 100 mM NaCl. The samples were then TEV digested to remove the leucine zippers and electrophoresed on a 13% non-denaturing gel in 25 mM Tris, 192 mM glycine buffer at pH 8.8 at 90V for 4 hours at 4° C. A leucine zippered MHC-I/TAPBPR sample (TEV−) was analyzed for comparison with the unzippered complex. Bovine albumin, and alcohol dehydrogenase, yeast was loaded in lane 1 as markers. (B) TAPBPR and TAPBPR/MHC-I complex was TEV digested and electrophoresed for comparison with the peptide loaded complex. The gels were stained with Coomassie blue (R250).

FIG. 8 provides a native gel shift assay of peptide binding to glycosylated MHC-I/TAPBPR, demonstrating that the complex is peptide-receptive, with the correct peptide specificity. SEC purified “unzippered” MHC-I/TAPBPR was incubated at a 5:1 molar ratio with TAX, MARTI NIH and P18-I10 peptides for one hour at room temperature prior to electrophoresis on a 12% non-denaturing gel in 25 mM Tris, 192 mM glycine buffer at pH 8.8 at 90V for 4 hours at 4° C. TAX and MART1 have high affinity for HLA-A*02:01, while NIH and P18-I10 are non-binders. The gels were stained with Coomassie blue (R250).

FIGS. 9A through 9F depict the design and biochemical analysis of native MHC-I/TAPBPR complexes. (A) CMV expression-cassette organization. (B) Chromatography of zippered and TEV-digested HLA-A*02:01/TAPBPR complexes on a S200 16/300 increase column (50 mM Tris, 100 mM, NaCl, running at a flow rate of 0.5 mL/min.). (C) Elution of un-zippered complex (line) at 27.5 minutes was confirmed by SDS PAGE. (D) PNGaseF-digested wild-type HLA-A02:01 and HLA-A02:01Δglycan (S88A) TAPBPR complex migration on a 12% SDS PAGE gel. (E) Native gel shift electrophoresis of HLA-A*02:01/TAPBPR complexes, showing pMHC-I formation in the presence of high-affinity peptides TAX9, MART-1 relative to the non-binding peptides NIH p29 and P18-I10. Gels were stained using InstantBlue (Expedeon). (F) Mass spectroscopy of recombinant MHC-I (HLA-A*02:01) with predominant N-glycan species ranked by spectral counts. MS2 spectrum of the predominant N86 glycopeptide indicating charge, composition and m/z for the individual fragment ions. Individual carbohydrate residues are shown according to the SNFG convention.

FIGS. 10A through 10D depict a summary of studies showing the specificity of peptide loading on CHO-derived HLA-A*02:01 and HLA-A*68:02. (A) Sequence logos of 9 mer epitopic peptides (IEDB database), ranked by frequency (Seq2logo). (B) Structure models of peptides GLLGIGILTV (SEQ ID NO:3), ETAGIGILTV (SEQ ID NO:5) and GLLGIGILTV (SEQ ID NO:3) bound to HLA-A*02:01 and HLA-A*68:02. A comparative modeling approach in the program Rosetta was used, with templates obtained from X-ray structures with PDB accession codes 4JFO, 3MRK, and 4JFF, respectively (Nerli and Sgourakis, bioRxiv, 2020.03.23.004176 (2020)). (C) Native gel shift assay showing pMHC-I formation in the presence of a panel of peptides with predicted affinities for HLA-A*02:01 and HLA-A*68:02 shown in Table 1. (D) Same assay as in (C) showing peptide binding on HLA-A*02:01 for a panel of SARS-CoV-2 epitopic peptides predicted using a structure-based algorithm (Nerli and Sgourakis, bioRxiv, 2020.03.23.004176 (2020)) (Table S1). As a positive control, we used the confirmed epitopic peptide NLVPMVATV (SEQ ID NO:45) from CMV, and as a negative control a non-specific (NS) peptide with sequence YPNVNIHNF (SEQ ID NO:7) (NIH p29). Native gels (12% polyacrylamide) were electrophoresed at 90V for 5 hrs at 4° C. before visualization with InstantBlue (Expedeon).

FIG. 11 depicts a summary of studies, showing that exchanged HLA-A*02:01 tumor antigens are recognized by their cognate T cell receptors. Top panel: DMF5+ cells were examined by flow cytometry following staining with 1μ/ml HLA-A*02:01 tetramers plus anti-CD8 FITC (BD) antibody. Tetramers were prepared using i) in vitro refolded HLA-A*02:01/MART-1, ii) empty HLA-A*02:01/TAPBPR complexes and iii) TAPBPR-exchanged HLA-A*02:01 with the specific MART-1 or iv) non-specific NY-ESO-1 peptide. Bottom panel: the complementary experiment using T cells expressing a NY-ESO-1 specific receptor. The approx. 9% tetramer-negative population of NY-ESO-1 cells, seen using both refolded and exchanged tetramers, corresponds to cells that have lost expression of the aβ TCR during culture. All flow cytometric analysis was performed using a BD LSR II instrument equipped with FACSDiva software (BD Biosciences) and FlowJo software (Ashland Oregon). Gating strategy is shown in FIG. 14.

FIGS. 12A and 12B show a structure modeling of the glycan moiety on the conserved Asn 86 residue of exemplary MHC-I molecule HLA-A*02:01. (A) HLA-A*02:01 in complex with the heteroclitic MART-1 peptide (PDB ID: 1JF1) modelled to show a biantennary disialated glycan at positon N86. (B) Electrostatic surface representation of the MHC-I molecule and glycan from the top view and side view. Solvent-accessible surface representation with electrostatic potential in the indicated ranges (−2 kcal/(mole) to +2 kcal/(mole) were calculated using CHARMM-GUI. All calculations were performed at 150 mM ionic strength, 298 Kelvin, pH 7.2, protein dielectric 2.0, and solvent dielectric 78.54. Electrostatic potentials are given in units of kT/e. A 1.4 Å solvent (probe) radius and 10.0 points/Å2 density was used to calculate molecular surfaces.

FIGS. 13A through 13C provide an analysis of an exemplary glycosylated MHC-I/chaperone complex as described herein, HLA-A*24:02/TAPBPR leucine-zippered complex. (a) Streptactin-purified complex was TEV digested and electrophoresed on a 12% SDS PAGE gel prior to size exclusion chromatography (SEC). (b) Gel electrophoresis of complex eluted from an SEC S200 16/300 increase column (50 mM Tris, 100 mM, NaCl, running at a rate of 0.5 mL/min). The complex peak (25-27.5 min) was collected. (c) Native gel electrophoresis of HLA-A*24:02/TAPBPR complex dissociation in the presence of relevant high affinity peptides (Nef 138_10 and PHOX2B) and irrelevant peptide (NIHp29).

FIGS. 14A through 14C provide a further flow cytometry analysis of the studies depicted in FIG. 11. (a) DMF5 cells are a HLA-A*02:01 restricted human lymphocyte line that express both the MART-1 specific TCR and CD8 co-receptor. For flow cytometry, dead-cells (PE-Cy5-A channel) were identified by treatment with ethanol followed by propidium iodide staining (b) Live singletons were selected by forward and side light scattering properties. The percentage of total population within each gate is indicated. (c) Titration of PE-HLA-A*02:01 MART-1 (WT) -and Δglycan (S88A) tetramers on DMF5 by flow cytometry with inset, 1.25 μg/mL tetramer, 1 μg/mL anti-human CD8-FITC (BD San Jose).

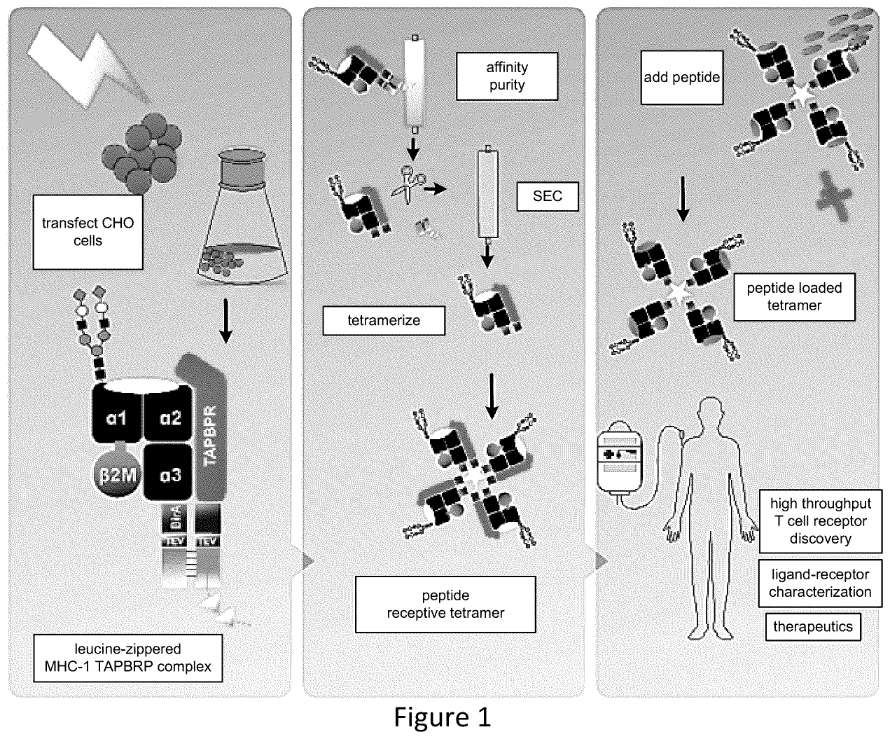

### DETAILED DESCRIPTION

### A. Overview

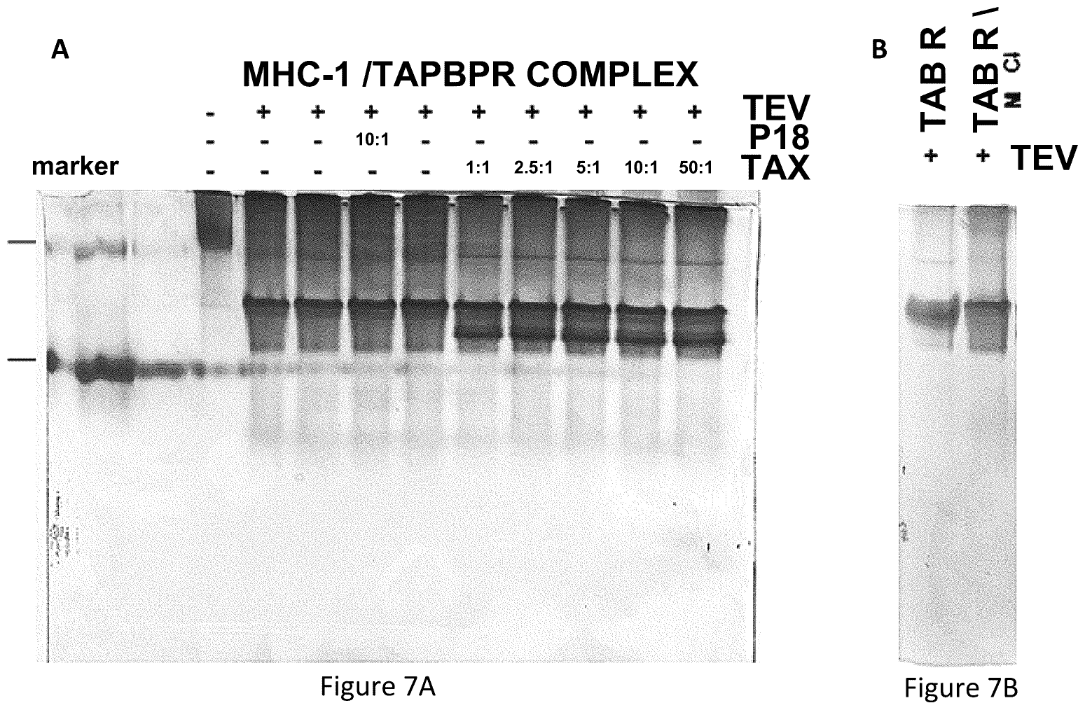

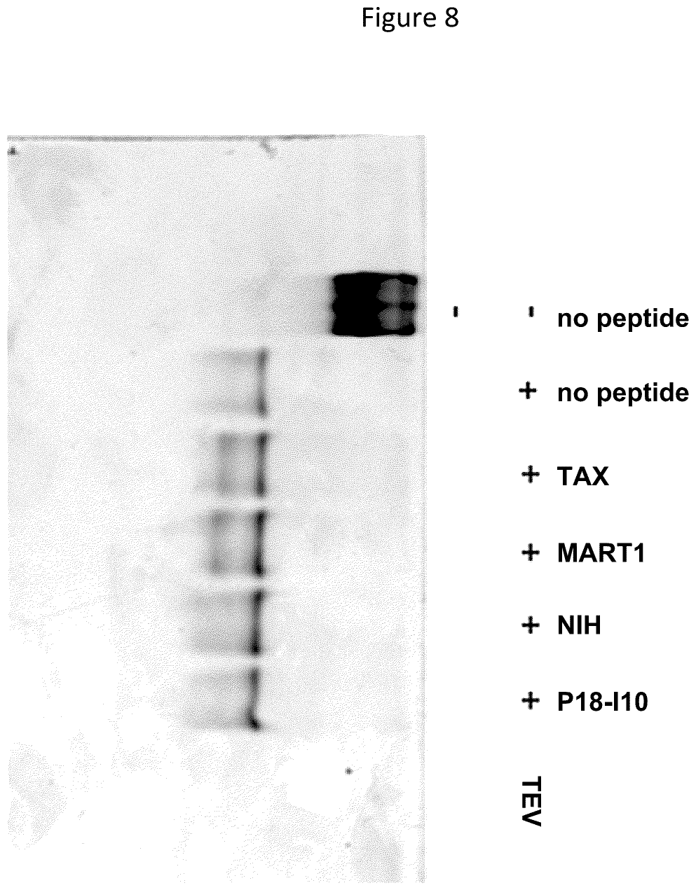

Disclosed herein are novel glycosylated peptide receptive MHC-I complexes that allow for efficient production of high affinity peptide (peptide of interest) MHC-I multimers (pMHC-I multimers). Such glycosylated peptide receptive MHC-I complexes include an MHC-I (e.g., a single-chain MHC-I) and are produced in mammalian expression systems (e.g., CHO and HEK cells) that allow for the glycosylation of the complexes at one or more amino acid positions (e.g., conserved N86 in HLA-A, HLA-B, and HLA-C). In certain embodiments, the glycosylated peptide receptive MHC-I complex further includes a TAPBPR. In some embodiments, the peptide receptive MHC-I complex includes a endogenous peptides, chaperones, or other proteins/peptides assocated with the peptide receptive complex. In certain embodiments, the glycosylated peptide receptive MHC-I complex is a glycosylated single chain MHC-I molecule capable of accepting a peptide of interest that is not associated with any other protein or peptide. The protein constructs used to make such glycosylated peptide receptive MHC-I complexes can include heterodimerization domains (e.g., leucine zipper domains) and/or one or more purification tags that facilitate purification of the complexes. The subject peptide receptive MHC-I complexes are capable of binding high-affinity peptides with the correct peptide specificity (FIGS. 7 and 8). Further, the peptide-receptive glycosylated MHC-I complexes can be used to produce pMHC multimer libraries for basic research, diagnostic and therapeutic applications, as described herein.

The compositions and methods described herein provide an efficient process for producing MHC tetramers than previous labor-intensive and inefficient methods.

Moreover, the use of glycosylated MHC-I molecules coexpressed in mammalian cells with the molecular chaperone TAPBPR results in a number of advantages. It provides native, peptide-receptive MHC-I complexes containing MHC-I molecules that are glycosylated at one or more native positions (e.g., the conserved N86). Upon multimerization and loading with high-affinity peptide, glycosylated peptide receptive MHC-I complexes allow stable antigen presentation in a physiologically relevant form of the MHC-I molecule.

Further, the multimers (e.g., tetramers) produced from the glycosylated peptide receptive MHC-I complexes provided herein advantageously allow for the identification of high-affinity T cell and natural killer cell receptors previously unidentified using traditional unglycosylated MHC tetramers, such as those produced in non-mammalian expression systems (e.g., Drosophila S2 or E. coli expression systems). TCRs identified using the MHC-I tetramers made using the complexes provided herein can provide important targets for both understanding antigen recognition processes, and the development of immunotherapies to combat bacterial and viral infections and cancers. Aspects of the glycosylated peptide receptive MHC-I complexes are further described in detail below.

### B. MHC Protein Constructs

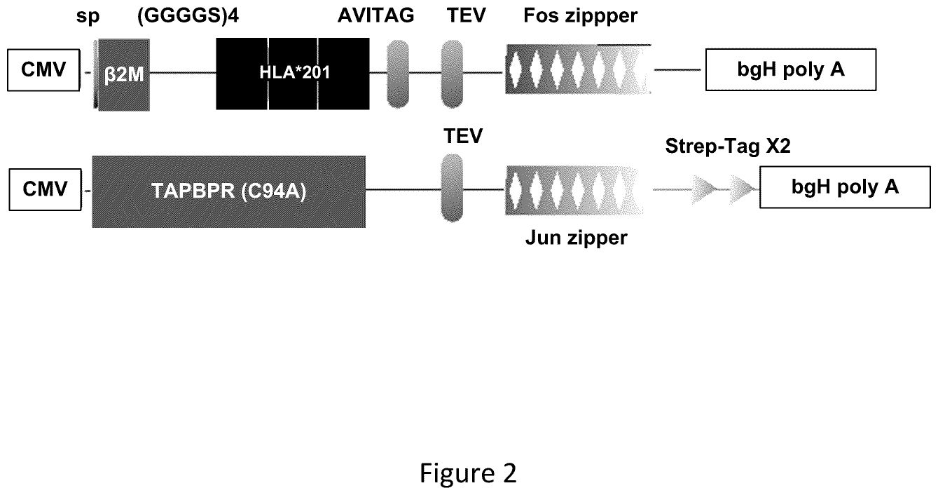

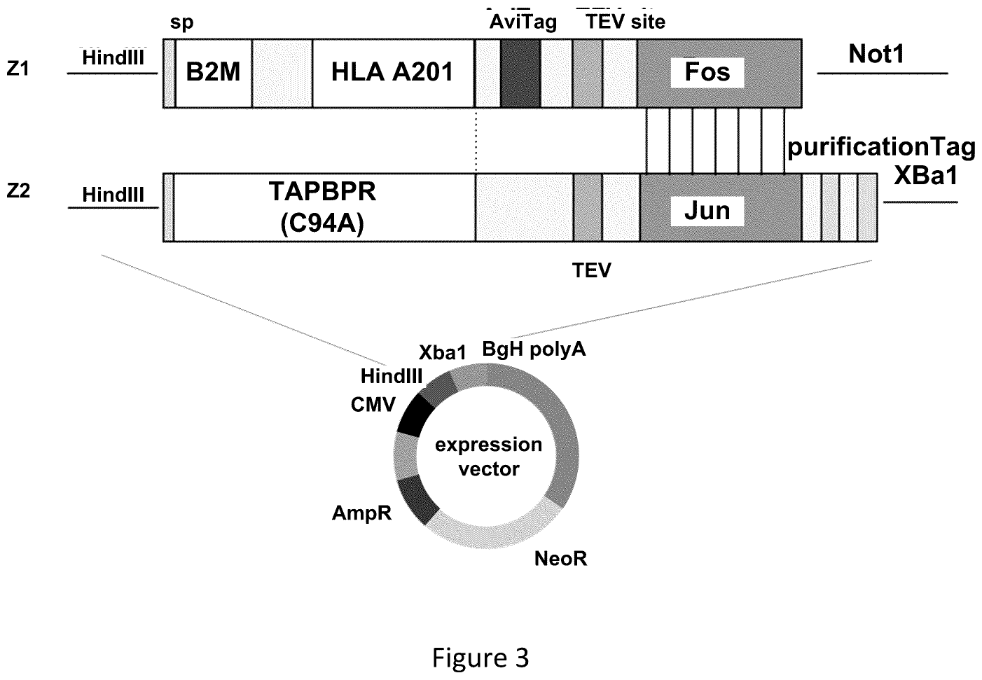

In one aspect, provided herein are MHC-I protein constructs that include: a) a MHC-I that includes a MHC-I heavy chain and a β2 microglobulin; b) a heterodimerization domain; and c) a protease cleavage site (see, e.g., FIGS. 2 and 3). Such MHC-I protein constructs, together with the chaperone protein constructs provided herein, are useful in making the subject glycosylated MHC-I/TAPBPR complexes.

The a) MHC-I, b) heterodimerization domain, and c) protease cleavage site of the MHC protein constructs provided herein are covalently linked from N- to C-terminus according to the following order: a) MHC-I, c) protease cleavage site, and b) heterodimerization domain. Any suitable linkers can be used to link the various parts of the MHC protein construct together, including those provided herein.

In related aspects, the MHC-I protein construct lacks a heterodimerization domain.

In some embodiments, the MHC-I protein construct further includes a d) multimerization tag that facilitates the formation of multimers (e.g., tetramers). Exemplary multimerization tags include, for example, tags that facilitate biotinylation such as AviTags. Biotinylated MHC-I protein constructs can be attached to a backbone (e.g., streptavidin) to form MHC multimers. In such embodiments, the parts of the MHC-I protein construct are covalently linked from N- to C-terminus according to the following order: a) single-chain MHC-I, d) multimerization tag, c) protease cleavage site, and b) heterodimerization domain (see, e.g., FIGS. 2 and 3).

Subject MHC-I protein constructs provided herein are made using any suitable technique including standard molecule biology and cloning techniques as described by Maniatis et al., “Molecular Cloning: A Laboratory Manual”, Cold Spring Harbor Laboratory, 1982, CSH, New York.

Nucleic acids encoding the MHC-I protein constructs and chaperone protein constructs described herein are coexpressed in a mammalian expression system (e.g., CHO or HEK cells). Expression in mammalian cells allow for the glycosylation of the single-chain MHC-I at one or more native glycosylation positions (e.g., N86). As used herein, a native glycosylation position refer to an amino acid position that is glycosylated in wild-type MHC-I. Such positions are referred to by a numbering convention based on the mature MHC-I molecule (i.e., without signal peptide) wherein amino acid position 1 is the first amino acid at the N-terminal of the mature MHC-I molecule, amino acid position 2 is the second amino acid from the N-terminal of the mature MHC-I molecule, etc.

As used herein, an “MHC class I,” “Major Histocompatibility Complex class I,” “MHC-I,” MHC I, and the like all refer to a member of one of two primary classes of major histocompatibility complex (MHC) molecules (the other being MHC class II) that are found on the cell surface of all nucleated cells in the bodies of jawed vertebrates. MHC class I molecules function to display peptide fragments of antigen to cytotoxic T cells, resulting in an immediate response from the immune system against a particular peptide antigen displayed within the peptide-binding groove of an MHC-I molecule.

MHC-I molecules are heterodimers that consist of two polypeptide chains: an α (heavy chain) and a β2-microglobulin (light chain). The two chains are typically linked noncovalently via interactions between the light chain and the α3 domain of the heavy chain and the floor of the α1/α2 domain. The heavy chain is polymorphic and encoded by an HLA gene, while the light chain is species-invariant and encoded by the Beta-2 microglobulin gene. The α3 domain is plasma membrane-spanning and interacts with the CD8 co-receptor of T cells. The α3-CD8 interaction holds the MHC-I molecule in place while the T cell receptor (TCR) on the surface of the cytotoxic T cell binds its syngeneic MHC-I ligand (or matched, in the sense that both the TCR and MHC-I are encoded in the same germline), and checks the displayed peptide for antigenicity. The α1 and α2 domains of the heavy chain fold to make up a groove for peptides to bind. MHC class I molecules bind peptides that, in most cases, are 8-10 amino acid in length.

In mice, MHC class I is called the “H-2 complex” or “H-2” and include the H-2D, H-2K and H-2L subclasses. In humans, MHC class I molecules include the highly polymorphic human leukocyte antigens HLA-A, HLA-B, HLA-C and the less polymorphic HLA-E, HLA-F, HLA-G, HLA-K and HLA-L. Each human leukocyte antigen (e.g., HLA-A) includes multiple alleles. For example, HLA-A includes over 2,430 non-redundant known alleles. Exemplary HLA-A alleles used in the protein constructs and methods described herein include, but are not limited to: HLA-A*02:01, HLA-A*24:02, HLA-A*68:01 or HLA-A*68:02.

In some embodiments, the MHC-I constructs provided herein include a single-chain MHC-I. Such single-chain MHC-I constructs include a MHC-I heavy chain covalently attached to a β2-microglobulin. In some embodiments of the MHC-I constructs, the single-chain MHC-I includes, from N- to C-terminus, MHC-I heavy chain-linker-β2 microglobulin. In another exemplary embodiment, the single-chain MHC includes, from N- to C-terminus, β2 microglobulin-linker-MHC-I heavy chain. In other embodiments, the MHC-I constructs include an MHC-I where the MHC-I heavy chain and β2 microglobulin are separate and not covalently attached by a linker.

Any suitable MHC-I heavy chain can be included in the MHC-I constructs provided herein. In some embodiments, the MHC-I heavy chain is an HLA-A heavy chain. In certain embodiments, the MHC-I heavy chain is an HLA-B heavy chain. In other embodiments, the MHC-I heavy chain is an HLA-C heavy chain. In an exemplary embodiment, the MHC heavy chain is an HLA-A*02:01, HLA-A*24:02, HLA-A*68:01 or HLA-A*68:02 allele heavy chain. In other embodiments, the MHC-I protein construct includes a mouse H-2. In certain embodiments, the H-2 is an H-2D, H-2K or H-2L. In exemplary embodiments, the H-2 is H-2D^{D} or H-2L^{D}. In some embodiments, the MHC construct include a variant of a wild-type MHC-I heavy chain. In particular embodiments, the variant MHC-I heavy chain has at least 80%, 85%, 90%, 91%, 92%, 93%, 94%, 95%, 96%, 97%, 98% or 99% sequence identity to a wild-type MHC-I heavy chain. Any suitable linker can be used to attach the MHC-I heavy chain to the β2 microglobulin. In certain embodiments, the linker is (GGGS)ₓ, (SEQ ID NO:46) wherein X is 1, 2, 3, 4, 5, 6, 7, 8, 9, 10. In an exemplary embodiment, the linker is (GGGS)₄ (SEQ ID NO:47).

In some embodiments, the MHC-I protein constructs provided herein include a heterodimerization domain that, upon binding to a heterodimerization domain of the chaperone protein construct, forms a stable “zippered” heterodimeric MHC-I/chaperone complex. Such stable “zippered” heterodimeric MHC-I/chaperone complexes are subsequently purified using any technique in the art. Any suitable heterodimerization domain that facilitate the heterodimerization of a MHC protein construct and chaperone protein construct can be used. In some embodiments, the heterodimerization domains favor the formation of the heterodimeric MHC-I/chaperone complex over homodimers that include two MHC-I protein constructs or two chaperone protein constructs. In some embodiments, the heterodimerization domains include coiled-coil heterodimerization domains. In certain embodiments, the heterodimerization domains are leucine zipper domains. In an exemplary embodiment, the leucine zipper domain is a Fos or Jun leucine zipper domain. In particular embodiments, the MHC-I protein construct includes a Fos domain and the chaperone protein construct includes a Jun domain. In other embodiments, the MHC-I protein construct includes a Jun domain and the chaperone protein construct includes a Fos domain.

The MHC-I protein constructs provided herein can include a protease cleavage site that facilitates the cleavage of the heterodimerization domain from the MHC-I protein construct after co-purification of the “zippered” heterodimeric MHC-I/chaperone complex. Any suitable protease cleavage site can be incorporated into the MHC-I protein construct. The protease that recognizes the protease cleavage site does not cleave the MHC-I protein construct at any site or in any domain other than the protease cleavage site. In an exemplary embodiment, the same protease cleavage site included in the MHC protein construct is also include in the chaperone protein construct. In such an embodiment, one protease is used to remove the heterodimerization domains on each of construct of the “zippered” heterodimeric MHC-I/chaperone complex to produce a mature “unzippered” peptide receptive MHC-I complex. Suitable cleavage sites include, but are not limited to enterokinase (DDDK)(SEQ ID NO:48), Factor Xa (IEGR/IDGR), Tobacco Etch Virus (ENLYFQS)(SEQ ID NO:30), thrombin (LVPRGS)(SEQ ID NO:49) and PreScission (LEVLFQGP)(SEQ ID NO:50), furin (Arg-X-X-Arg)(SEQ ID NO:52) and genenase (Arg-X-(Lys/Arg)-Arg)(SEQ ID NO:53) protease cleavage sites. In an exemplary embodiment, the protease cleavage site is a Tobacco Etch Virus (TEV) protease cleavage site.

In some embodiments, the MHC-I protein construct can further include e) one or more purification tags at its carboxyl terminal that facilitate purification of the MHC-I protein construct and/or a “zippered” heterodimeric MHC-I/chaperone complex. In such embodiments, the parts of the MHC protein construct are covalently linked from N- to C-terminus according to the following order: a) single-chain MHC-I, d) protein tag, c) protease cleavage site, b) heterodimerization domain and e) purification tag(s). In some embodiments, the purification tag allows for affinity purification of the “zippered” heterodimeric MHC-I/chaperone complexes from cell culture medium. Suitable purification tags that can be included in the chaperone protein construct include, but are not limited to, histidine tags, Strep-tags®, MYC-tags and HA-tags. In an exemplary embodiment, the purification tag is a Strep-tags®.

Following purification and “unzippering” of the glycosylated heterodimeric MHC-I/chaperone complexes by protease cleavage the resulting peptide receptive MHC-I complexes can be used to form multimers (e.g., tetramers or Dextramers®). To facilitate multimerization, the MHC-I protein constructs provided herein optionally include a protein tag that is capable of being biotinylated (see FIGS. 2 and 3). Such proteins tags can be located between the single-chain MHC-I and the protease cleavage site. Upon cleavage of the protease site, the protein tag is located at the C-terminus of the mature “unzippered” glycosylated peptide receptive MHC-I complex (see, e.g., FIG. 2). Biotinylation of glycosylated peptide receptive MHC-I/complexes via protein tags allow for the attachment of such complexes to modular backbones (e.g., streptavidin or dextran) to form multimers (e.g., tetramers or Dextramers®). Such multimers can be stored or directly loaded with high-affinity peptides to form pMHC-I multimers and used in applications wherein such multimers are useful, as disclosed herein (e.g., T cell repertoire analysis, receptor ligand characterization studies and T cell stimulation).

In an exemplary embodiment, the MHC-I protein construct includes from N- to C-terminus orientation: a) a signal peptide; b) B2-microglobulin; c) an MHC-I heavy chain; d) a protein tag for multimerization; e) a protease cleavage site; and f) a leucine zipper heterodimerization domain (e.g., Fos or Jun domain) (see, e.g., FIGS. 2 and 3). In such an embodiment, a)-f) are covalently linked using suitable peptide linkers.

### C. Chaperone Protein Constructs

In another aspect, provided herein are chaperone protein constructs that include: a) a chaperone; b) a heterodimerization domain; and c) a protease cleavage site. Such chaperone protein constructs (e.g., TAPBPR protein constructs), together with the MHC protein constructs provided herein, are useful in making the subject glycosylated MHC-I/chaperone complexes.

The a) a chaperone; b) a heterodimerization domian; and c) a protease cleavage site of the chaperone protein constructs provided herein are covalently linked from N- to C-terminus according to the following order: a) chaperone, c) protease cleavage site, and b) heterodimerization domain. Any suitable linkers can be used to link the various parts of the chaperone construct together, including those provided herein. Subject MHC protein constructs provided herein are made using any suitable technique including standard molecule biology and cloning techniques as described by Maniatis et al., “Molecular Cloning: A Laboratory Manual”, Cold Spring Harbor Laboratory, 1982, CSH, New York

In some aspects, the chaperone protein construct lacks a heterodimerization domain, including aspects where both the chaperone protein construct and the MHC-I protein construct lack heterodimerization domains.

In some embodiments, the chaperone included in the chaperone protein construct is a Tapasin Binding Protein Related (TAPBPR). TAPBPR protein includes a signal sequence, three extracellular domains comprising a unique membrane distal domain, an IgSF (immunoglobulin superfamily) V domain and an IgC1 domain, a transmembrane domain, and a cytoplasmic region. (Boyle et al., PNAS 110 (9) 3465-3470 (2013); incorporated by reference herein).

When co-expressed in mammalian cells, the chaperone protein constructs and MHC-I protein constructs provided are capable of forming “zippered” glycosylated heterodimeric MHC-I/chaperone complexes via the heterodimerization domains included in each construct. As discussed above, any suitable heterodimerization domain that facilitates the formation of the “zippered” glycosylated heterodimeric MHC-I/chaperone complexes over homodimeric species can be used. In some embodiments, the heterodimerization domains include coiled-coil heterodimerization domains. In certain embodiments, the heterodimerization domains are leucine zipper domains. In an exemplary embodiment, the leucine zipper domain is a Fos or Jun leucine zipper domain. In particular embodiments, the MHC-I protein construct includes a Fos domain and the chaperone protein construct includes a Jun domain. In other embodiments, the MHC-I protein construct includes a Jun domain and the chaperone protein construct includes a Fos domain.

The chaperone protein constructs provided herein include a protease cleavage site that facilitates the cleavage of the heterodimerization domain from the chaperone protein construct after co-purification of the zippered MHC protein construct/chaperone protein construct heterodimer. Any suitable protease cleavage site can be incorporated into the chaperone protein construct. The protease that recognizes the protease cleavage site does not cleave the chaperone protein construct at any site or in any domain other than the protease cleavage site. In an exemplary embodiment, the same protease cleavage site included in the chaperone protein construct is also include in the MHC-I protein construct. In such an embodiment, one protease is used to remove the heterodimerization domains on each of construct of the “zippered” glycosylated heterodimeric MHC-I/chaperone complex, thereby “unzippering” the complex and resulting in a peptide receptive MHC-I . Suitable cleavage sites include, but are not limited to enterokinase (DDDK)(SEQ ID NO:48), Factor Xa (IEGR/IDGR), Tobacco Etch Virus (ENLYFQS)(SEQ ID NO:30), thrombin (LVPRGS)(SEQ ID NO:49) and PreScission (LEVLFQGP)(SEQ ID NO:50), furin (Arg-X-X-Arg)(SEQ ID NO:52) and genenase (Arg-X-(Lys/Arg)-Arg)(SEQ ID NO:53) protease cleavage sites. In an exemplary embodiment, the protease cleavage site is a Tobacco Etch Virus (TEV) protease cleavage site.

In some embodiments, the chaperone protein construct further includes: d) one or more purification tags that facilitate the co-purification of the zippered MHC protein construct/chaperone protein construct heterodimers (also referred to herein as glycosylated MHC-I/chaperone complexes). In such embodiments, the parts of the chaperone protein construct are covalently linked from N- to C-terminus according to the following order: a) chaperone, c) protease cleavage site, b) heterodimerization domain, and d) purification tag(s). Any tag that allows for co-purification of the zippered MHC-I protein construct/chaperone protein construct heterodimer can be included in the chaperone protein construct. In some embodiments, the purification tag allows for affinity purification of the “zippered” glycosylated heterodimeric MHC-I/chaperone complexes from cell culture supernatant. Suitable purification tags that can be included in the chaperone protein construct include, but are not limited to, histidine tags, Strep-tags®, MYC-tags and HA-tags. In an exemplary embodiment, the purification tag is a Strep-tags®.

Following purification and protease treatment of the “zippered” glycosylated heterodimeric MHC-I/chaperone complexes to yield peptide receptive MHC-I complexes, the peptide receptive MHC-I complexes can be multimerized, as discussed above. Such multimers can be stored or directly loaded with high-affinity peptides (pMHC-I multimers). Once loaded with high-affinity peptides, the glycosylated pMHC-I multimers can be used in applications wherein such multimers are useful, as disclosed herein (e.g., T cell repertoire analysis, receptor ligand characterization studies and T cell stimulation). In other embodiments, the mature “unzippered” glycosylated heterodimeric MHC-I/chaperone complexes are loaded with high-affinity peptides before the formation of multimers to form glycosylated pMHC-I complexes. In an exemplary embodiment, the chaperone protein construct includes from N- to C-terminus orientation: a) a TAPBPR chaperone; b) a protease cleavage site; c) a leucine zipper heterodimerization domain (e.g., Fos or Jun domain), and d) one or more purification tags (see, e.g., FIGS. 2 and 3). In such an embodiment, a)-d) are covalently linked using suitable linkers.

### D. Glycosylated MHC/Chaperone Mammalian Expression Systems

In another aspect, provided herein are expression vectors that include a polynucleotides encoding one or more of the MHC protein constructs and/or chaperone protein constructs provided herein. Expression vector compositions that include a) a first polynucleotide encoding a MHC-I protein construct described herein; and b) a second polynucleotide encoding a chaperone protein construct described herein are also provided. In preferred embodiments, the expression vector is a mammalian expression vector. In exemplary embodiments, each of the first polynucleotide and second polynucleotide are included in the same expression vector (see FIG. 3). In other embodiments, the first polynucleotide and second polynucleotide are included in different expression vectors. In some embodiments wherein the first and second polynucleotides are included in the same expression vector, expression of the MHC-I protein construct and chaperone protein construct is controlled by the same promoter. In other embodiments, the expression of the MHC-I protein construct and chaperone construct are controlled by different promoters. In an exemplary embodiment, the promoter is a cytomegalovirus (CMV) or SV40 promoter.

As discussed herein, the MHC-I protein constructs and chaperone protein constructs are expressed using a mammalian expression system and/or cell line that advantageously allows for post-translational glycosylation of the MHC-I protein at one or more native positions (e.g., N86). Such glycosylated MHC-I proteins, when multimerized, allow for the identification of high-affinity T cell and natural killer (NK) cell receptors previously unidentified using traditional unglycosylated pMHC-I tetramers produced in non-mammalian expression systems (e.g., Drosophila S2 or E. coli expression systems).

Cultured mammalian cell lines that are useful for making the glycosylated peptide receptive MHC-I complexes and tetramers described herein include, but are not limited to, Chinese hamster ovary (CHO), COS, HEK and HeLa cell lines. In certain embodiments, the protein constructs provided herein are expressed using a CHO-K1 cell line.

### E. Methods of Making Glycosylated Peptide Receptive MHC-I Complexes

The MHC-I protein constructs, chaperone protein constructs and mammalian expression systems provided herein can be used to make peptide receptive MHC-I complexes, wherein the MHC-I molecule is glycosylated at one or more native glycosylation position (e.g., conserved N86 of MHC-I).

In such a method, a mammalian host cell (e.g., CHO or HEK cell) is first provided that includes an expression vector having a first nucleic acid encoding a MHC-I protein construct described herein. In some embodiments, wherein an MHC-I/chaperone construct is desired, the host cell further includes an expression vector having a second nucleic acid encoding a chaperone protein construct described herein. In other embodiments, each of the first nucleic acid and second nucleic acid are included in separate expression vectors in the mammalian host cell. The mammalian host cell is cultured under suitable conditions where the MHC-I protein construct and chaperone protein construct are co-expressed and the constructs undergo post-translation glycosylation at one or more native glycosylation positions (e.g., conserved N86 of a MHC-I molecule). As described herein, the MHC-I protein construct and chaperone protein construct each include a heterodimerization domain (e.g., leucine zipper domains) that facilitate the heterodimerization of the MHC-I protein construct and the chaperone protein construct to form “zippered” heterodimeric MHC-I/chaperone complexes. In some embodiments, the heterodimerization domains include coiled-coil heterodimerization domains. In certain embodiments, the heterodimerization domains are leucine zipper domains. In an exemplary embodiment, the leucine zipper domain is a Fos or Jun leucine zipper domain. In particular embodiments, the MHC-I protein construct includes a Fos domain and the chaperone protein construct includes a Jun domain. In other embodiments, the MHC-I protein construct includes a Jun domain and the chaperone protein construct includes a Fos domain.

The “zippered” heterodimeric MHC-I/chaperone complexes are purified from the cellular supernatant using any suitable methods. In some embodiments, a purification tag is included in the C-terminal of one or both of the MHC-I protein construct and chaperone protein construct. Suitable purification tags that can be included in the chaperone protein construct and/or MHC-I protein construct include, but are not limited to, histidine tags, Strep-tags®, MYC-tags and HA-tags. In an exemplary embodiment, the chaperone protein construct includes a Strep-tag®. The culture medium containing the purification tagged “zippered” heterodimeric MHC-I/chaperone complexes is applied to an affinity column that binds the complexes via the purification tags. In some embodiments, the affinity column includes streptavidin or Strep-Tactin®, which allows for the capture of zippered” heterodimeric MHC-I/chaperone complexes that include a Strep-tag®. Following capture of the zippered” heterodimeric MHC-I/chaperone complexes, the complexes are eluted from the column and subsequently contacted with a protease (e.g., TEV protease) that cuts each of the MHC protein construct and chaperone protein construct at a protease cleavage site, thereby removing the heterodimerization domains and “unzippering” the mature glycosylated MHC-I/chaperone complexes. The “unzippered” glycosylated MHC-I/chaperone complexes can be purified away from the cleaved heterodimerization domains, for example, by size exclusion chromatography. Such constructs are capable of receiving a high affinity peptide of interest and are can therefore be termed glycosylated peptide receptive MHC-I complexes can be used to form MHC tetramers contacted with high-affinity peptides to form pMHC multimers (e.g., tetramers). The peptide receptive MHC-I chaperone constructs can be complexed with a chaperone protein, but need not be.

In still other embodiments, MHC-I protein constructs and chaperone protein constructs are engineered without a heterodimerization domain and coexpressed in mammalian cells. The chaperone constructs act upon the MHC-I protein constructs catalytically (e.g. where the MHC-I protein construct and the chaperone protein construct do not form a stable complex) to transform the MHC-I protein constructs into peptide receptive MHC-I complexes that can be purified and loaded with peptide as described herein. No protease treatment is necessary in this particular embodiment.

### F. Glycosylated MHC-I/TAPBPR Multimers

The glycosylated peptide receptive MHC-I complexes provided herein can be used to form glycosylated peptide receptive MHC-I multimers and/or glycosylated multimers of an MHC-I complexed with a peptide of interest (called a pMHC-I herein). Such multimers (e.g., tetramers or Dextramers®) can allow for the identification of high-affinity T cell and natural killer (NK) cell receptors previously unidentified using traditional unglycosylated MHC-I tetramers produced in non-mammalian expression systems (e.g., Drosophila S2 or E. coli expression systems).

In some embodiments, glycosylated peptide receptive MHC-I multimers can be produced by attaching biotinylated glycosylated peptide receptive MHC-I complexes to a backbone. Biotinylation of the glycosylated peptide receptive MHC-I complexes can be performed by contacting the complexes with biotin in the presence of a biotin ligase enzyme.

In some embodiments, the backbone is a streptavidin backbone. In certain embodiments, the backbone is an avidin backbone. In other embodiments, the backbone is a dextran backbone.

In some embodiments, contacting the glycosylated peptide receptive MHC-I complex with high-affinity peptide to form pMHC-I complexes occurs prior to multimerization. The pMHC-I complexes can then be biotinylated and attached to backbones to form pMHC-I multimers. In exemplary embodiments, peptide deficient MHC class I/chaperone complexes are biotinylated first and then attached to a backbone (e.g., a streptavidin, avidin or dextran backbone), thereby forming peptide deficient MHC class I/chaperone multimers (e.g., tetramers). Such peptide receptive MHC class I multimers can be used for the large scale production of pMHC-I multimers comprising one or more peptides of interest by contacting the peptide receptive MHC class I multimers with the one or more peptides of interest. For example, in one embodiment, aliquots of the peptide receptive MHC-I multimers are contacted with different peptides of interest, thereby forming a library of pMHC-I multimers. After loading of the pMHC-I multimers with peptides of interest, the resulting loaded pMHC-I multimers can be washed to remove any free chaperones, labels (e.g., nucleic acid barcodes) or excess peptides of interest not bound in the pMHC-I complexes. Following such a washing step, the exchanged pMHC-I multimers can be stored (e.g., 4° C. for several weeks) or used immediately. In some embodiments, the free chaperones, labels and/or peptides of interest are removed by spin column dialysis.

In some embodiments, the MHC-I protein construct includes a protein tag that facilitates multimerization. Such protein tags are capable of being biotinylated, thereby allowing the attachment of the MHC-I protein constructs to backbones to form multimers. In some embodiments, the protein tag included in the MHC-I protein construct includes one or more amino acid residues that can be biotinylated. in an exemplary embodiment, the protein tag includes exactly one amino acid residue that can be biotinylated. In certain embodiments, the amino acid residue is a lysine residue. In particular embodiments, the protein tag is an AviTag (GLNDIFEAQKIEWHE) (SEQ ID NO:34) that includes one lysine residue.

In some embodiments, the glycosylated pMHC-I multimer is a dimer. In some embodiments, the pMHC-I multimer is a trimer. In preferred embodiments, the glycosylated pMHC-I multimer is a tetramer. In one embodiment, the multimer is a dextramer. Dextramers include ten glycosylated MHC-I complexes attached to a dextran backbone. Dextramers allow for the detection, isolation, and quantification of antigen specific T cell populations due to an improved signal-to-noise ratio not present in prior generations of multimers. See, e.g., Bakker and Schumacher, Current Opinion in Immunology 17(4): 428-433 (2005); and Davis et al., Nature Reviews Immunology 11:551-558 (2011).

In some embodiments, the glycosylated pMHC-I multimer is a glycosylated pMHC-I tetramer that includes four glycosylated pMHC-I molecules, wherein the four glycosylated pMHC-I molecules are each attached to a streptavidin backbone. In some embodiments, each of the four glycosylated pMHC-I molecules are biotinylated and attached to one of the four biotin binding subunits of the streptavidin backbone. In one embodiment each of the four glycosylated pMHC-I molecules is glycosylated at least one native glycosylation site. In an exemplary embodiments, each of the four glycosylated pMHC-I molecules is glycosylated at N86.

In some embodiments, the four glycosylated pMHC-I complexes each include a glycosylated single-chain MHC-I protein construct that includes an MHC-I heavy chain covalently linked to a β2 microglobulin. The single chain MHC-I protein construct is complexed with a peptide of interest. Any suitable MHC-I heavy chain allele can be included in the single-chain MHC-I protein construct. In some embodiments, the single-chain MHC-I protein construct includes an HLA-A heavy chain. In certain embodiments, the single-chain MHC-I protein construct includes an HLA-B heavy chain. In other embodiments, the single-chain MHC-I protein construct includes an HLA-C heavy chain. In an exemplary embodiment, the single-chain MHC-I protein construct includes an HLA-A01 or HLA-A02 allele heavy chain. In an exemplary embodiment, the MHC heavy chain is an HLA-A*02:01, HLA-A*24:02, HLA-A*68:01 or HLA-A*68:02 allele heavy chain. In other embodiments, the single chain MHC-I protein construct includes a mouse H-2. In certain embodiments, the H-2 is an H-2D, H-2K or H-2L. In exemplary embodiments, the H-2 is H-2D″ or H-2LD. In some embodiments, the single-chain MHC-I protein construct include a variant of a wild-type MHC-I heavy chain. In particular embodiments, the variant MHC-I heavy chain has at least 80%, 85%, 90%, 91%, 92%, 93%, 94%, 95%, 96%, 97%, 98% or 99% sequence identity to a wild-type MHC-I heavy chain. Any suitable linker can be used to attach the MHC-I heavy chain to the β2 microglobulin. In certain embodiments, the linker is (GGGS)ₓ,(SEQ ID NO:46) wherein X is 1, 2, 3, 4, 5, 6, 7, 8, 9, 10. In an exemplary embodiment, the linker is (GGGS)₄(SEQ ID NO:47).

In certain embodiments, at least one of the glycosylated MHC-I molecules of the multimer is complexed with a chaperone molecule. In some embodiments, one, two, three, four or more of the glycosylated MHC-I molecules of the multimer are each complexed with a chaperone molecule. In some embodiments, the chaperone molecule is TAPBPR. In exemplary embodiments, the glycosylated MHC-I molecules of the tetramer are each loaded with a peptide of interest (i.e., glycosylated pMHC-I tetramers).

In some embodiments, the backbone of the pMHC-I multimer is conjugated with a detectable label (e.g., a fluorophore or a radiolabel) that allow the multimer to be detected in various applications. In certain embodiments, the detectable label is as fluorophore. See, e.g., Nepom et al., J Immunol 188 (6) 2477-2482 (2012). In one embodiment, the detectable label is a radiolabel. In certain embodiments, the backbone includes a barcode (e.g., a nucleic acid barcode) that allows the glycosylated pMHC-I multimer to be used in large scale high throughput processes. See, e.g., Bentzen et al., Nature Biotechnology 34(1): 1037-1045 (2016). In an exemplary embodiment, unique barcodes are used for each of the different peptides of interest included in the glycosylated pMHC-I multimers, thereby allowing for the tracking, sorting and identification of particular glycosylated pMHC-I multimers in high throughput applications. In particular embodiments, each barcode includes a unique nucleotide sequence.

In some embodiments, the glycosylated pMHC-I multimer is coupled to a toxin (e.g., saporin). Such pMHC-I multimer conjugates can be used to modulate or deplete specific T cell populations. See, e.g., Maile et al., J. Immunol. 167: 3708-3714 (2001); and Yuan et al., Blood 104: 2397-2402 (2004).

### G. Methods of Using Tetrameric Glycosylated MHC-I/TAPBPR Complexes

In certain embodiments, the pMHC-I multimer produced from the glycosylated peptide receptive MHC-I complex provided herein is a pMHC-I tetramer. pMHC-I tetramers provided herein can be used to study pathogen immunity, for the development of vaccines, in the evaluation of antitumor response, in allergy monitoring and desensitization studies, and in autoimmunity. See, e.g., Nepom et al., J Immunol 188 (6) 2477-2482 (2012); and Davis et al., Nature Reviews Immunology 11:551-558 (2011).

In some embodiments, the pMHC-I multimers are used to characterize T cell (e.g., CD8 T cell) responses to a vaccine, including, but not limited to influenza, yellow fever, tuberculosis, coronavirus, (e.g. SARS-CoV-2), and HIV/SIV vaccines. In an exemplary embodiment, the vaccine is a cancer vaccine. In particular embodiments, the cancer vaccine is melanoma or chronic myeloid leukemia. In such embodiments, a sample (e.g., a blood sample) of a vaccinated patient is contacted with one or more of the subject pMHC-I multimers that include one or more peptide of interests derived from the vaccine to identify and monitor antigen specific T cells that are produced in response to the vaccine.

Peptide-MHC-I multimers provided herein can also be used to isolate and enrich particular antigen specific T cells for therapeutic use. See, e.g., Cobbold et al., J. Exp. Med. 202: 379-386 (2006); and Davis et al., Nature Reviews Immunology 11:551-558 (2011). In this particular application, patient samples are contacted with sortable pMHC-I multimers that include a peptide antigen of interest and a label that allows for sorting (e.g., a fluorophore or nucleic acid label). Antigen specific T cells that bind the pMHC-I multimer are subsequently isolated and purified, for example, using flow cytometry or similar cell sorting and identification techniques.

In certain embodiments, the pMHC-I multimers provided herein are used for epitope mapping. In this method, a plurality of pMHC-I multimers that include different peptides derived from an antigen of interest (e.g., a tumor antigen) are contacted with a sample from a subject. Antigen specific T cells are detected and the corresponding epitope peptide sequences are identified any technique known in the art, include, for example, flow cytometry and cell sorting techniques. See, e.g., Bentzen et al., Nat Biotechnol. 34(10):1037-1045 (2016).

In some embodiments, the pMHC-I multimers provided herein are used to determine a T cell profile of one or more subjects. In such an embodiment, a sample from a subject is contacted with a library of pMHC-I multimers that include a library of peptides of interest and a detectable label. Identification of antigen specific T cells that bind particular peptides of interest presented in the context of the pMHC-I multimers is achieved using the detectable label. The methods described herein allow for the large scale production of pMHC-I multimer libraries that can in turn be used for high throughput T cell profiling.

In another aspect, the pMHC-I multimers are used therapeutically for the targeted elimination of particular antigen specific T cells in a subject. In one embodiment, the pMHC-I multimers are conjugated to a cytotoxic agent or a toxin. When administered to a subject, the pMHC-I multimer conjugates attach to and facilitate the elimination of particular antigen specific T cells.

Peptide-MHC class I multimers used in the methods described herein can be tracked and detected using any suitable techniques including, but not limited to, techniques utilizing detectable labels and nucleic acid barcodes that allow identification of particular pMHC class I multimers. In addition, T cells of interest isolated in such methods can also be identified using similar techniques.

T cells of interest that interact with pMHC-I multimers can be isolated using any suitable technique including, for example, flow cytometry techniques. Isolated T cells and corresponding peptide-MHC class I multimers can then be characterized using any suitable method, for example, the ECCITE-seq method as explained below in conjunction with 10×Genomics CHROMIUM SINGLE CELL IMMUNE PROFILING SOLUTION™ with FEATURE BARCODING™ technology. This method incorporates a cellular barcode into cDNA generated from both tetramer oligos and TCR mRNA, thus the pairing of cellular barcodes can connect TCR sequences and other mRNAs with pMHC-I multimers specificities.

### EXAMPLES

### Example 1: Glycosylated MHC-I/TAPBPR Complexes

Peptide exchange technologies are central to the generation of high throughput pMHC-multimer libraries currently used for probing polyclonal TCR repertoires. To date, only non-glycosylated MHC molecules produced in E. coli and refolded in vitro have been available for library construction. Although glycosylation is not essential for peptide loading, the biological significance of a single highly conserved glycan on MHC Class I molecules, remains to be determined.

Expression of MHC-I complexes with TAPBPR in mammalian cells provides native, peptide-receptive MHC-I/TAPBPR complexes that are glycosylated. For example, such complexes would include the critical glycosylation at the conserved N86 in HLA-A, HLA-B, and HLA-C. Upon multimerization and loading with high-affinity peptides, as described in Overall et al., BioRxiv (2019)), incorporated by reference herein, these complexes allow stable antigen presentation in a physiologically relevant form of the molecule that can in turn be used to identify high-affinity T cell and natural killer (NK) cell receptors. The disclosed method is modular, and is applicable to a range of applications, including immune repertoire characterization on patient samples, as outlined in detail below. Furthermore, the high level expression of peptide-receptive MHC-I molecules in mammalian cells can result in therapeutic and applications such as vaccines.

Previous work (Overall et al supra) made use of MHC refolding protocols from inclusion bodies expressed in E. coli and TAPBPR expressed in S2 Drosophila cells. Such methods of producing pMHC-I complexes require refolding and purification. In addition, MHC-I molecules produced in E. coli lack a glycan post-translational modification at the conserved N86 residue. The glycan modification is known to provide stability to the MHC-I. The glycan is at the face of the MHC which is known to interact with T cell and natural killer (NK) cell receptors, and could therefore play important roles in physiologically relevant immune recognition.

So disclosed herein is a mammalian expression system, including engineered protein constructs for the preparation of peptide-receptive MHC molecules in complex with the molecular chaperone TAPBPR which can be used directly for the preparation of MHC multimer libraries, and other applications.

The class I molecules of the Major Histocompatibility Complex (MHC) play a pivotal role in orchestrating an adaptive immune response by alerting the immune system to the presence of developing infections and tumors in the body. Immune surveillance is achieved through the display of short (8-11 residue long) peptides derived from viral proteins (or mutated oncogenes) via a tight interaction with the MHC-I peptide-binding groove. Such peptide/MHC-I protein complexes are assembled inside the cell and displayed on the surface of all antigen-presenting cells where they can interact with specialized receptors on T cells and natural killer (NK) cells. The MHC-I proteins are extremely polymorphic (more than 13,000 different alleles have been identified in the human population to date), and each allele can display an estimated 1,000-10,000 different peptides, which makes the characterization of specific T cell responses against a panel of known peptide epitopes a daunting task, further challenged by the fact that typical T cell receptor affinities for their cross-reactive pMHC ligands are low (e.g., in the micromolar range).

The use of multivalent, fluorescent pMHC-I multimers was pioneered by Altman and Davis in 1996 to stain T cells (Altman J D et al., Science 274, 94-96 (1996); Altman J D & Davis M M, Curr Prot Immunol Ch. 17, Unit 17.3 (2003); both of which are incorporated by reference). Cells that recognize a specific peptide/MHC multimer can be identified and sorted using flow cytometry, and their receptors can be sequenced in subsequent steps. Peptide-MHC-I (pMHC-I) tetramers have revolutionized experimental immunology and the development of new therapies, leading to a breadth of discoveries (Doherty P C, J Imunol 187, 5-6 (2011); incorporated by reference herein).

However, the preparation of properly conformed pMHC molecules via in vitro refolding of inclusion bodies expressed in E. coli, requires a laborious, multi-step process that is highly inefficient (typical refolding yields are <5% by weight). Moreover, all MHC molecules expressed in E. coli lack the functionally relevant post-translational glycosylations that are required for proper immune surveillance function (Garboczi D N et al., PNAS 89, 3429-3433 (1992); Barber et al., J Immunol 156, 3275-3284 (1996); both of which are incorporated by reference herein). Specifically, a conserved glycan at residue N86 of the MHC-I protein, which is located at a site near the TCR recognition surface, is not present in E. coli expressed MHC-I. This limits the application of refolded tetramers to identify high-affinity T cell receptors and natural killer cell receptors and results in a more limited TCR repertoire than would be present in vivo. The missing TCRs can include important targets for a number of applications including the study of antigen recognition processes, and the development of immunotherapies to combat bacterial and viral infections and cancer.

The use of MHC-I molecules expressed in mammalian cells as a high-affinity complex with the molecular chaperone TAPBPR (McShan A C et al., Nat Chem Biol 14, 811-820 (2018); incorporated by reference herein) results in a number of advantages. It provides native, peptide-receptive MHC-I complexes containing the critical glycosylation at the conserved N86. Upon multimerization and loading with high-affinity peptides as described in Overall et al supra, peptide receptive MHC-I complexes allow stable antigen presentation in a physiologically relevant form of the molecule towards the identification of high-affinity T cell and natural killer cell receptors. The use of affinity tags attached to the recombinant proteins results in specific binding for MHC-I molecules results in fewer non-specific peptides contaminating the library.

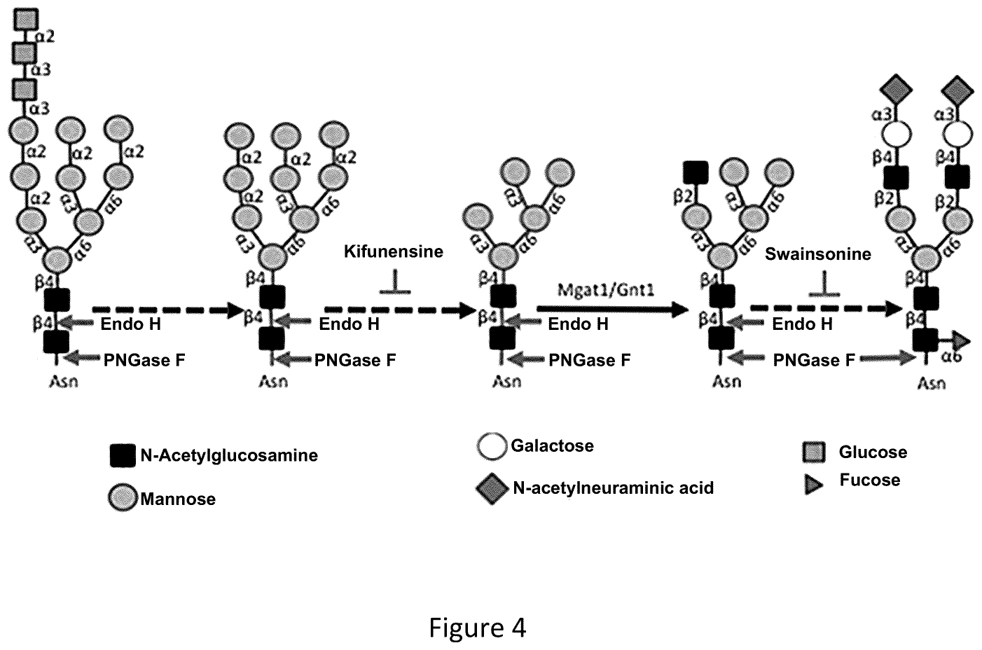

Efficient expression of a peptide receptive MHC-I complex in mammalian cells has the potential to simply the workflow to prepare peptide loaded tetramers for T cell analysis. Previously disclosed methods involve the refolding of E. coli inclusion-body expressed protein. In addition, MHC molecules produced in prokaryotic systems lack glycosylation. Human MHC-I (HLA A, B and C) are highly polymorphic but the N86 PNGS shows considerable conservation across phylogeny (Grossberger D and Parham P, Immunogenetics 36, 166 (1992); incorporated by reference herein). Glucose trimming of ER associated core N-glycan Glc₃Man₉GlcNAc₂ facilitates proper interactions with the lectin chaperones calnexin and calreticulin, but the function of glycans subsequently added by the N-linked glycosylation pathway is yet to be determined (Barber L D et al., J Immunol 156, 3275-3284 (1996) and Ryan S O and Cobb B A, Semin Immunopathol 34, 424-441 (2012); both of which are incorporated by reference herein). A simplified version of the mammalian N-linked glycan pathway is shown in FIG. 4. Described herein is a method of preparing native, peptide-receptive MHC-I complexes that include the highly conserved N86 glycan.

Based on an early report of proteolysis of MHC-I heavy chain by complement CIS enzyme between the α2 and α3 heavy chains (Erikson H and Nissen M H, Biochem Biophys Res Commun 187, 832-838 (1989)); incorporated by reference herein), initial small-scale expression trials for the production of MHC-I/TAPBPR were performed in a C1s−/− CHOK1s knockout (Li S et al., Biotechnol Bioeng, doi: 10.1002/bit.27016 (2019); incorporated by reference herein). Constructs used herein are shown in FIG. 3.

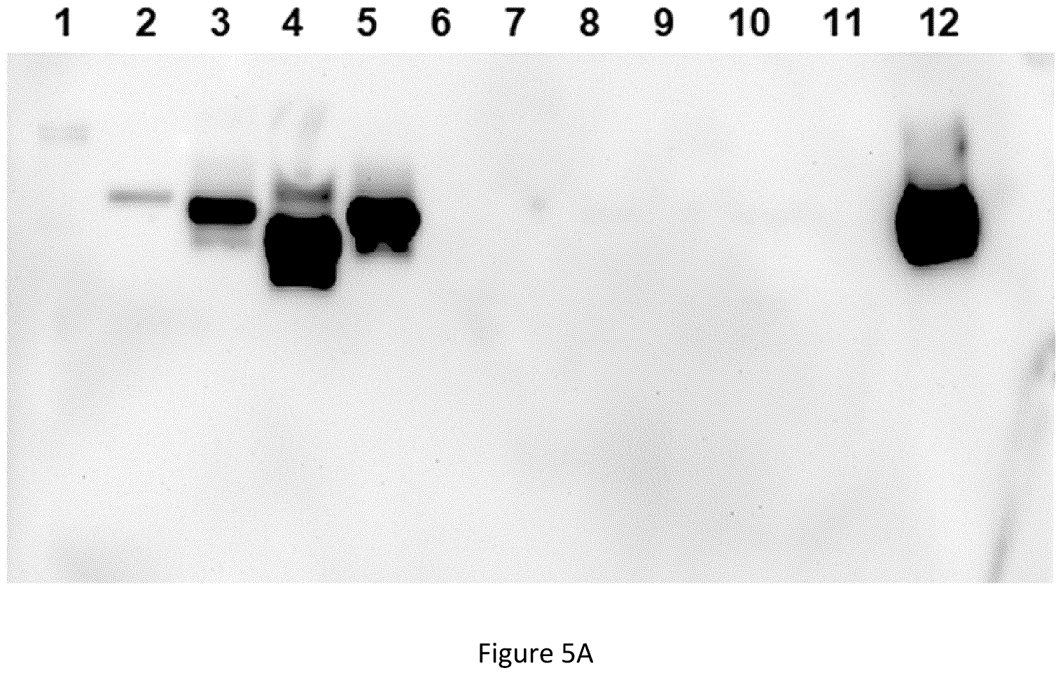

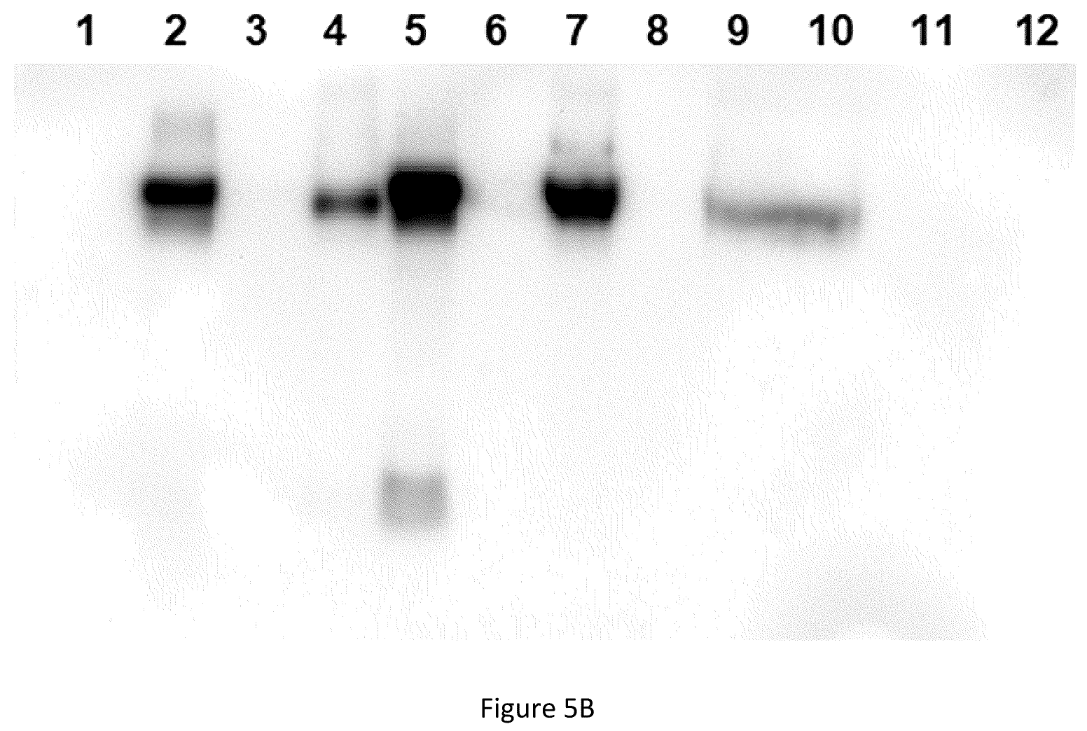

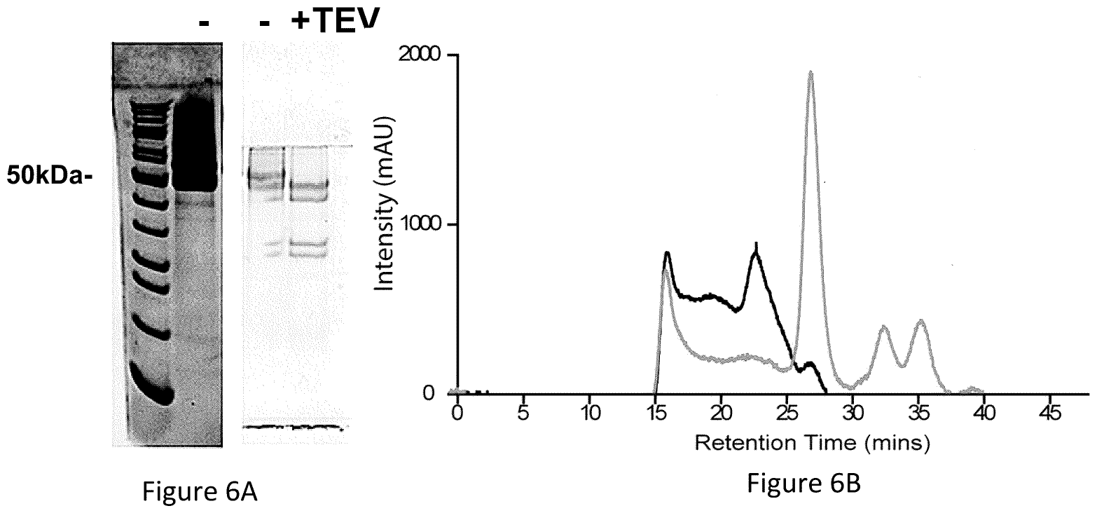

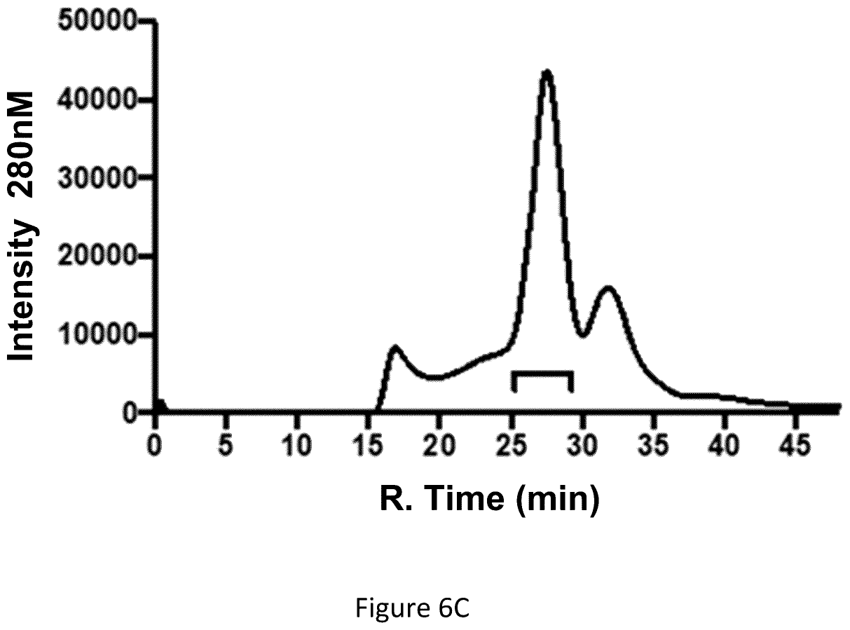

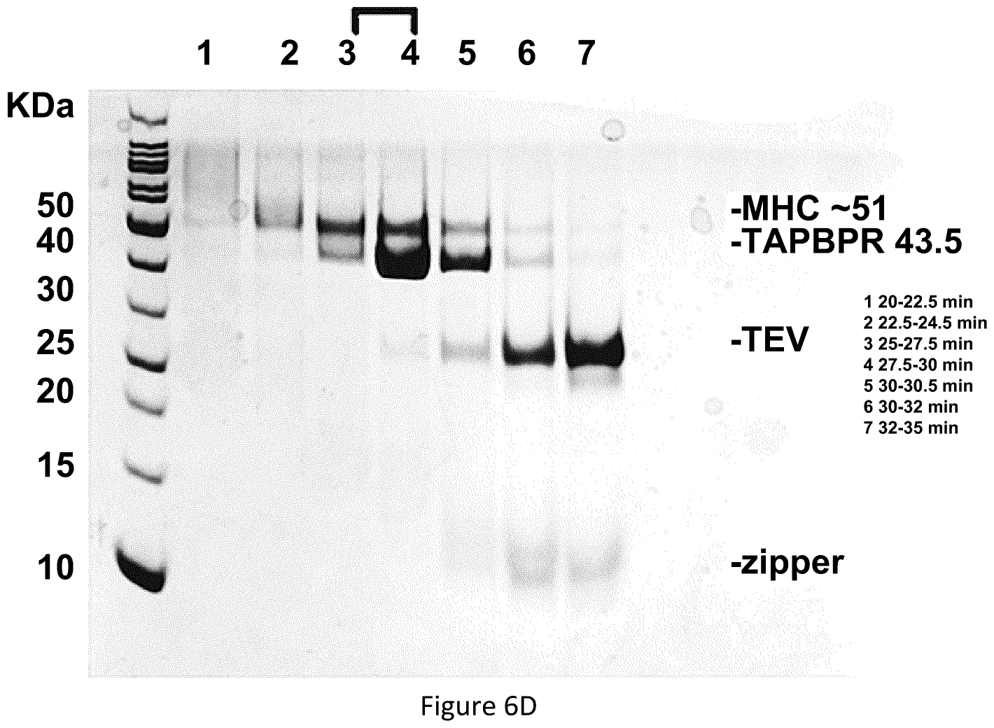

Small scale (C400/15 mL) 5 day transfections yielded between 10-30 mg/L of purified complex, prior to any optimization. Typical cell growth is shown in FIG. 5. Harvesting the cultures at day 5 ensured cell health (viability >95%) but early protein harvest reduces potential protein yield. Western blots of culture supernatant confirmed secretion of TAPBPR and MHC-I by transfected cells. Co-transfection plasmids expressing a single-chain MHC-I-B2M protein and a TAPBPR protein with complementary JUN and FOS leucine zipper sequences (O'Shea E K et al., Science 245, 646-648 (1989) and Kalandadze A et al., J Biol Chem 271, 20156-20162 (1996)); both of which are incorporated by reference herein) resulted in the production of a leucine-zippered complex. The complex was purified via a streptactin-tag located on the carboxy-terminal of TAPBPR, which resolves on a 12% SDS PAGE gel as two bands at approximately 51 and 43.5 kDa. (Coomassie stained gel FIG. 6A). After TEV digestion, a distinct shift in molecular mass is evident on the SDS PAGE (FIG. 6A) coincident with zipper cleavage. When “unzippered” and “zippered” complex are compared on an analytical size exclusion gel filtration column (FIG. 6B), the zippered complex peaks at approximately 24 minutes, while the TEV digested molecule peaks at 26.5-27 minutes. S200 increase size exclusion chromatography after TEV cleavage (FIGS. 6C and 6D) permitted isolation of the MHC-I/TAPBPR complex, which was subsequently concentrated and stored at 4° C. LS-MS analysis determined the un-zippered (TEV cleaved) TAPBPR to be 43.5 kDa, as predicted by amino acid composition. Incubation of the zippered complex with a molar equivalent or excess of peptide with a high affinity for HLA-A*02:01, resulted in the characteristic native gel-shift of the MHC-I band from diffuse to highly defined (FIG. 7). MHC-I/TAPBPR complexes fully purified prior to assay (i.e., TEV digested and size exclusion purified) behaved in an identical manner (FIG. 8). High affinity peptide (MARTI and TAX) resulted in a band shift while non-binders NIH and P10-I18 did not, confirming the hypothesis that we had produced and purified a glycosylated peptide-receptive MHC-I complex.

The glycosylated peptide receptive MHC-I complexes produced in CHO cells can be tetramerized and loaded with peptide, in the same manner as bacterially expressed and refolded (and therefore glycan free) peptide receptive MHC-I complexes.

MART-1 loaded tetramers produced via the disclosed methods (i.e., “single-chain complex” produced in CHO cells) can specifically bind to Jurkat/MA cells expressing the DMF5 T cell receptor that is specific for HLA-A*02:01/MART-1. Tetramers loaded with the irrelevant NYESO peptide, however, did not bind to such Jurkat cells expressing the DMF5 T cell receptor. Similarly, tetramers produced via the disclosed methods loaded with NYESO peptide can specifically bind to Jurkat cells expressing a unique T cell receptor specific for HLA-A*02:01/NYESO. Tetramers loaded with MART-1 peptide, however, did not bind non-specifically to such Jurkat cells expressing NYESO T cell receptor. The single-chain MHC tetramers produced using the disclosed methods bind T cells in a specific manner, similar to those tetramers produced using conventional methods.

### Materials and Methods

Molecular cloning. Vectors designated (Z1 and Z2) are suitable for transient and stable high level expression of secreted proteins (particularly proteins that are engineered to include leucine zipper domains) in mammalian cells. The recombinant proteins comprising the leucine zippers can be purified directly from tissue culture supernatant using a StrepTrap HP affinity column (GE Healthcare, Chicago IL). The leucine zipper domain can be removed by TEV protease digestion, and peptide receptive MHC-I molecules purified by size exclusion chromatography Construct Z1 expresses a single MHC-I single- chain gene that expresses a recombinant protein comprising the human B2M sequence (UniProtKB P61769) a flexible linker sequence (Hansen et al., 2009), and the ecto-domain of MHC-I HLA* 0201 exons 1-4. Construct Z2 expresses TAPBPR (UniProtKB Q9BX59-1) and a Strep-Tactin® tag. Standard molecular protocols were used to construct expression vectors. Briefly, synthetic, codon optimized B2M_MHC-I and TAPBPR genes were purchased from IDT (Coralville, IA) and cloned independently into a CMV driven expression cassette within a plasmid vector. Plasmids were propagated in the DH5 alpha strain of E. coli, and purified using an endotoxin free PureLink extraction kit (Life Technologies, Thermo Fisher, Carlsbad, CA). DNA sequencing was carried out at the University of California at Berkeley Core Sequencing facility using Sanger chain termination sequencing. The complete mature protein sequences are provided herein.

Cells and antibodies. CHO-K1 cells were obtained from ATCC (ATCC, Manassas, VA) and adapted to suspension culture by serial passage in suspension (CHO-K1s). A CHOK1s variant (CHO-K1s C1S−/−) was provided by Dr. Phil Berman (Li S et al., Biotechnol Bioeng, doi: 10.1002/bit.27016 (2019); incorporated by reference herein). HEK293F cells were obtained from Life Technologies (Thermo Fisher Carlsbad, CA). Anti-TAPBPR antibodies were purchased from Life Technologies (Thermo Fisher Carlsbad, CA) or raised by immunization of rabbits immunized using a Complete Freund's Adjuvant/Incomplete Freund's Adjuvant (CFA/IFA) protocol (Pocono Rabbit Farms, AAALAC #926, Canadensis, PA) with TAPBPR produced in CHO-K1s cells as an antigen. Anti-B2M macroglobulin antibodies were purchased from R & D Systems (Minneapolis, MN). Flow cytometry antibodies were purchased from BD Biosciences (San Jose, CA).

Cell culture conditions. Stocks of suspension adapted CHO-K1s, 293 HEKF, and CHO-K1s C1s−/−cells were maintained in shake flasks (Corning, Corning NY) using a Kuhner ISF1-X shaker incubator (Kuhner, Birsfelden, Switzerland). For normal cell propagation shake flasks cultures were maintained at 37° C., 8% CO₂, and 135 rpm. TCR β-chain deficient Jurkat-MA T cells expressing the DMF5 TCR recognizes Melan-A epitope MART-1 bound to HLA-A*02:01, were grown in DMEM supplemented with 10% heat inactivated FBS, 25 mM HEPES pH 7, 2 μM β-mercaptoethanol, 2 mM L-glutamine, 100 U/mL penicillin/streptomycin and 1×non-essential amino acids. All supplements were obtained from Life Technologies (Carlabad CA) unless stated otherwise. Static cultures were maintained in 96 or 24 well cell culture dishes and grown in a Sanyo incubator (Sanyo, Moriguchi, Osaka, Japan) at 37° C. and 5% CO₂.

Cell culture media. For normal CHO-K1s cell growth, cells were maintained in BalanCD CHO Growth A (Irvine Scientific, Santa Ana, CA) supplemented with 0.1% pluronic acid, 8 mM GlutaMax and 1×Hypoxanthine/Thymidine (Thermo Fisher, Life Technologies, Carlsbad, CA). 293 HEK (Freestyle) cells were maintained in Freestyle 293 cell culture media (Life Technologies, Carlsbad, CA). For CHO cell protein production, the cells were maintained at 32° C. (24 hours after transfection) in in BalanCD CHO Growth A medium supplemented with 0.1% pluronic acid, 2 mM GlutaMax and 1×H/T (Thermo Fisher, Life Technologies, Carlsbad, CA), and fed daily with MaxCyte CHO A Feed which is comprised of 0.5% Yeastolate, B D, Franklin Lakes, NJ; 2.5% CHO-CD Efficient Feed A, 2 g/L Glucose (Sigma-Aldrich, St. Louis, MO) and 0.25 mM GlutaMax).

Cell counts and growth calculation. All cell counts were performed using a TC20™ automated cell counter (BioRad, Hercules, CA) with viability determined by trypan blue (Thermo Fisher, Life Technologies, Carlsbad, CA) exclusion. Cell-doubling time in hours was calculated using the formula: (((time₂−time₁)×24)×ln (2)/(ln (density₂)−ln (densityl)).

Electroporation. Electroporation was performed using a MaxCyte STX scalable transfection system (MaxCyte Inc., Gaithersburg, MD) according to the manufacturer's instructions, using aseptic technique. Cells were maintained at >95% viability prior to transfection, and sub-cultured one day prior to transfection. The day of transfection, cells were pelleted at 250 g for 10 minutes, and then re-suspended in MaxCyte EP buffer (MaxCyte Inc., Gaithersburg, MD) at a density of 2×10⁸ cells/mL. Transfections were carried out in the OC-400 processing assembly (MaxCyte Inc., Gaithersburg, MD) with a total volume of 400 μL and 8×10⁷ total cells. Plasmid DNA in endotoxin-free water was added for a final concentration of 300 μg of DNA/ml. The processing assemblies were then transferred to the MaxCyte STX electroporation device and appropriate conditions (CHO protocol) were selected using the MaxCyte STX software. Following completion of electroporation, the cells in Electroporation buffer were removed from the processing assembly and placed in 125 mL Erlenmeyer cell culture shake flasks (Corning, Corning NY). The flasks were placed into 37° C. incubators with no agitation for 40 minutes. Following the rest period, pre-warmed OPTI-CHO media (Thermo Fisher, Invitrogen, Carlsbad, CA) supplemented with 0.1% pluronic acid, 2 mM GlutaMax and 1×H/T, was added to the flasks for a final cell density of 4×10⁶ cells/mL. Flasks were then moved the Kuhner shaker and agitated at 135 rpm.

ImmunoBlot. Proteins (from cell supernatant and cytoplasmic lysate) were electrophorized on 12% SDS gels in MOPS gel running buffer (Thermo Scientific, Waltham, MA). For Immunoblot, proteins were electrophoresed, transferred to a PDVF membrane, then probed with a polyclonal rabbit anti-TAPBPR antibody or a murine anti-B2M followed by an affinity purified secondary HRP conjugated anti-species antibody (Jackson ImmunoResearch, West Grove, PA) and visualized using an Innotech FluoChem2 system (Genetic Technologies Grover, MO).

Peptide Receptive MHC-I Complex Purification. Culture media was harvested and pre-cleared by centrifugation at 250 g for 10 minutes. The media was adjusted to contain 25 mM Tris pH 8, 1mM EDTA and 27 mg/L of avidin and filtered (0.22 micron) before affinity purification on a StrepTrap HP affinity column (GE Healthcare, Chicago IL). Bound protein was washed with 10 column volumes of wash buffer (25 mM Tris pH 8, 100 mM NaCl, 1 mM EDTA) and eluted with 2.5 mM desthiobiotin/wash buffer. The complex was concentrated from approximately 6 mL to 0.5 mL on a 30 kD cutoff MicrosepAdvance filter (Pall, New York, NY), and digested overnight with TEV (Tobacco Etch Protein) in TEV cleavage buffer (25 mM Tris pH 8, 100 mM NaCl 1 mM EDTA, 3 mM/0.3 M glutathione redox buffer at 4° C. Complex was recovered by gel filtration (SEC) on a Superdex 200 10/300 increase column (GE Healthcare, Chicago IL) at a flow rate of 0.5 mL/min in 50 mM Tris pH 7.5 buffer containing 100 mM NaCl at room temperature. MHC-I/TAPBPR complexes eluted at 26.5-27 minutes GM didpeptide was (10 mM) was added to the running buffer during chromatography.

LC-MS. The molecular mass of TEV digested TAPBPR was determined by HPLC separation on a Higgins PROTO300 C4 column (5 μm, 100 mm×21 mm) followed by electrospray ionisation performed on a Thermo Finnigan LC/MS/MS (LQT) instrument. Peptides were identified by extracting expected m/z ions from the chromatogram and deconvoluting the resulting spectrum in MagTran.

Native gel shift assay of peptide binding to empty complex. Peptide-receptive MHC-I complexes were incubated with the indicated molar ratio of relevant (TAX or MART1) or irrelevant (P18-I10 or NIH) peptide for 1 h at room temperature at pH 7.5 in Tris buffer with 50 mM NaCl. Samples were electrophoresed at 90 V on a 12% polyacrylamide gel in 25 mM T IS pH 8.8, 192 mM glycine, at 4° C. for 4.0 hours and developed using InstantBlue (Expedeon San Diego, CA).

Tetramer formation. The procedure for production of peptide loaded tetramers using TAPBPR mediated exchange is described in Overall et. al., (2019) (referenced above). Briefly, SEC purified (unzipped) peptide receptive MHC-I complex molecules were biotinylated via an AviTag (GLNDIFEAQKIEWHE) (SEQ ID NO:34) on the MHC-I molecule using biotin ligase (BirA) (Avidity.com Co), according to the manufacturer's instructions. Biotinylated MHC-I/TAPBPR complex was buffer exchanged into PBS pH 7.4 using a PD-10 desalting column. Biotinylation was confirmed by SDS-PAGE in the presence of excess streptavidin. Tetramerization of empty-MHC-I/TAPBPR was performed by adding a 2:1 molar ratio of biotinylated MHC-I/TAPBPR to streptavidin-PE or streptavidin-APC (Prozyme Hayward, CA) in five additions over 1 h on ice. Peptide-receptive MHC-I tetramers were then contacted with peptides of interest by adding a 20-molar excess of peptide to each well and incubating for 1 hour. A solution of 8M biotin (to block any free streptavidin sites) was added and incubated for a further 1 h at room temperature. After exchange, tetramers were transferred to 100 kDa spin columns (Amicon, Millipore, Burlington, Ma) and washed with 1000 volumes of PBS to remove TAPBPR and excess peptide. After washing, exchanged tetramers were pooled and stored at 4° C. for up to 3 weeks.

Flow cytometry. Tetramer analysis was carried out as described in Overall et al. (2019) supra by staining 2×10⁵ Jurkat/MA cells transduced with the DMF5 receptor specific to the MART-1, with an anti-CD8a mAb (BD Biosciences) and 1 μg/mL HLA-A02:01/MART-1 tetramer or HLA-A02:01/TAX tetramer for 1 h on ice, followed by two washes with 30 volumes of FACS buffer (PBS, 1% BSA, 2 mM EDTA). All flow cytometric analysis was performed using a BD LSR II instrument equipped with FACSDiva software (BD Biosciences).

For EC50 determination. Tetramer concentrations were calculated based on total amount of pMHC-I at the time of exchange. Titrations were performed on the appropriate cell line in duplicate in two independent experiments. The percentage of tetramer+ T cells was measured relative to the staining achieved at the highest concentration tested within each experiment. EC50 values were calculated by fitting a Boltzmann sigmoidal function to the data with the lower constraint set to 0 and upper constraints set to 95 for B4.2.3 and 28 for DMF5 in GraphPad Prism 7.

Differential scanning Fluorimetry (DSF). To measure thermal stability of pMHC-I complexes, 2.5 μM of protein was mixed with 10×Sypro Orange dye in matched buffers (20 mM sodium phosphate pH 7.2, 100 mM NaCl) in MicroAmp Fast 96 well plates (Applied Biosystems) at a final volume of 50 μL. DSF was performed using an Applied Biosystems ViiA qPCR machine with excitation and emission wavelengths at 470 nm and 569 nm respectively. Thermal stability was measured by increasing the temperature from 25° C. to 95° C. at a scan rate of 1° C./min. Melting temperatures (Tm) were calculated in GraphPad Prism 7 by plotting the first derivative of each melt curve and taking the peak as the Tm Determination of Tm values of TAPBPR exchanged molecules additionally required subtraction of the TAPBPR melt curve from the curve obtained for the complex, then calculating the first derivative. This procedure, on average, enhanced the Tm values calculated for TAPBPR exchanged pMHC-I complexes by 1.5° C., compared to refolded and photo-exchanged pMHC-I complexes. All samples were analyzed in duplicate and the error is represented as the standard deviation of the duplicates analyzed independently.

### Example 2: Production of Soluble pMHC-I Complexes in Mammalian Cells Using the Molecular Chaperone TAPBPR

Current approaches for generating MHC Class-I proteins with peptides of interest (pMHC-I) for diagnostic and therapeutic applications are limited by the inherent instability of empty MHC-I molecules. Using the properties of the chaperone TAP Binding Protein Related (TAPBPR), a robust method has been developed to produce properly conformed, peptide-receptive molecules in Chinese Hamster Ovary cells at high yield, completely bypassing the requirement for laborious refolding from inclusion bodies expressed in E. coli. Purified peptide receptive MHC-I/complexes can be prepared for multiple human allotypes, and exhibit complex glycan modifications at the conserved Asn 86 residue. As a proof of principle, both HLA allele-specific peptide binding, and MHC-restricted antigen recognition by T cells for a panel of HLA-A*02:01 epitopic peptides predicted from the SARS-COV-2 genome were demonstrated. The system disclosed herein provides a facile, high-throughput approach to probe polyclonal TCR repertoires against their cognate pMHC-I antigens.

### Introduction

The Class-I proteins of the Major Histocompatibility Complex (MHC-I) play a pivotal role in orchestrating immune responses through their interactions with specialized receptors on T cells and Natural Killer (NK) cells (Germain and Margulies, Annu. Rev. Immunol., 11, 403-450 (1993); Jiang et al., Adv. Exp. Med. Biol., 1172, 21-62 (2019)). Immune surveillance by αβ T cell receptors (TCRs) is achieved through the display of short (8-11 residue long) peptides derived from viral proteins (or mutated oncogenes) via tight capture within the MHC-I peptide-binding groove as an obligate protein complex (Rossjohn et al., Annu. Rev. Immunol., 33, 169-200 (2015)). MHC-I molecules are assembled on the endoplasmic reticulum (ER) from component heavy and β2 microglobulin (β2m) light chains and loaded with peptides in the context of a multi-subunit membrane complex (Cresswell et al., Immunol. Rev., 172, 21-28 (1999)). Interactions of nascent MHC-I with molecular chaperones (tapasin and TAPBPR) select for high-affinity peptides to ensure the prolonged stability and immunogenicity of resulting peptide/MHC-I (pMHC-I) complexes (Blum et al., Annu. Rev. Immunol., 31, 443-473 (2013)). As an additional quality control step, glucose trimming of the ER-associated Glc3Man9GlcNAc2 moiety found at the conserved Asn 86 (N86) residue ensures that only correctly folded molecules are trafficked further along the antigen processing pathway towards the cell surface (Wearsch et al., roc. Natl. Acad. Sci. U.S.A., 108, 4950-4955 (2011)). Although tapasin is an integral part of the ER-anchored peptide loading complex, TAPBPR is found throughout the ER and cis-Golgi network and has independent, auxiliary functions in MHC-I quality control (Neerincx et al., eLife, 6 (2017)) and in shaping the displayed peptide repertoire (Boyle et al., Proc. Natl. Acad. Sci. U.S.A., 110, 3465-3470 (2013); Hermann et al., eLife, 4 (2015); Hermann et al., Tissue Antigens, 85, 155-166 (2015)).

Detecting and quantifying antigen-specific TCRs during the course of disease, treatment or immunization was revolutionized by the use of multivalent, fluorescent, pMHC-I complexes (Altman et al., Science, 274, 94-96 (1996); Hadrup and Schumacher, Cancer Immunol. Immunother., 59, 1425-1433 (2010)). Empty MHC-I molecules are unstable and highly prone to aggregation, so pMHC-I proteins are commonly produced by in vitro refolding of light and heavy chain components, derived from E. coli inclusion bodies, in the presence of large molar excess of a synthetic peptide which involves a laborious multi-step process with typical yields of <5% by weight (Garboczi et al., Proc. Natl. Acad. Sci., 89, 3429-3433 (1992)). There have been considerable efforts to develop peptide-exchange methods, including photolabile peptides (Bakker et al., Proc. Natl. Acad. Sci. U.S.A., 105, 3825-3830 (2008)), dipeptide catalysts (Saini et al., Proc. Natl. Acad. Sci. U.S.A., 112, 202-207 (2015)), thermal exchange (Luimstra et al., J. Exp. Med., 215, 1493-1504 (2018)) or disulfide-linked MHC-I molecules (Moritz et al., Sci. Immunol., 4(37):eaav0860 (2019)). All these methods make use of refolded MHC-I molecules, lacking important post-translational modifications which are likely to influence peptide repertoire selection, and T cell and NK cell recognition. Methods for producing pMHC-I complexes in mammalian cells using covalently linked peptides as single-chain (Jurewicz et al., Anal. Biochem., 584, 113328 (2019)) or fused antibody-pMHC-I constructs (Schmittnaegel et al., Mol. Cancer Ther., 15, 2130-2142 (2016)) have been described, however both approaches necessitate cleavage of the bound peptide for exchange to occur, which results in low protein yields.

The use of molecular chaperones for peptide exchange applications was first explored in the context of Tapasin (Chen and Bouvier, EMBO J., 26, 1681-1690 (2007)). TAPBPR, known to stabilize the empty MHC-I peptide-binding groove in a widened conformation (Jiang et al., Adv. Exp. Med. Biol., 1172, 21-62 (2017); Thomas and Tampé, Science, eaao6001 (2017)) and to promote peptide exchange in vitro (Morozov et al., Proc. Natl. Acad. Sci. U.S.A., 113, E1006-E1015 (2016)), offers an attractive alternative to Tapasin from a biochemical perspective and has been exploited to load MHC-I molecules with peptides directly on the cell surface, independently of the peptide-loading complex (Ilca et al., Proc Natl Acad Sci U.S.A. 115(40), E9353-E9361 (2018); Ilca et al., eLife:7 (2019)). A detailed characterization of the TAPBPR catalytic cycle (McShan et al., Nat Biochem 14(8), 811-820 (2018)) has recently been leveraged to develop a high-throughput exchange methodology for multiple murine and human MHC-I allotypes expressed in E. coli and refolded with an exchangeable peptide (Overall et al., Nat. Commun., 11, 1-13 (2020)). Here, the chaperone function of TAPBPR was explored to develop a similar approach for producing soluble pMHC-I complexes, using a mammalian protein expression system. This approach was to engineer suitable MHC-I and TAPBPR constructs with a cleavable heterodimeric leucine-zipper, a system which enables the production of pMHC-I complexes of desired peptide specificities at mg quantities. Recombinant MHC-I/TAPBPR complexes produced in mammalian cells bypass the requirements and restrictions of the peptide loading complex, are subject to standard eukaryotic post-translational modification, and can be readily loaded with peptides towards functional, biochemical and structural characterization of interactions with their cognate immune receptors.

### Results

### Generating Properly Conformed MHC-I Molecules in CHO Cells

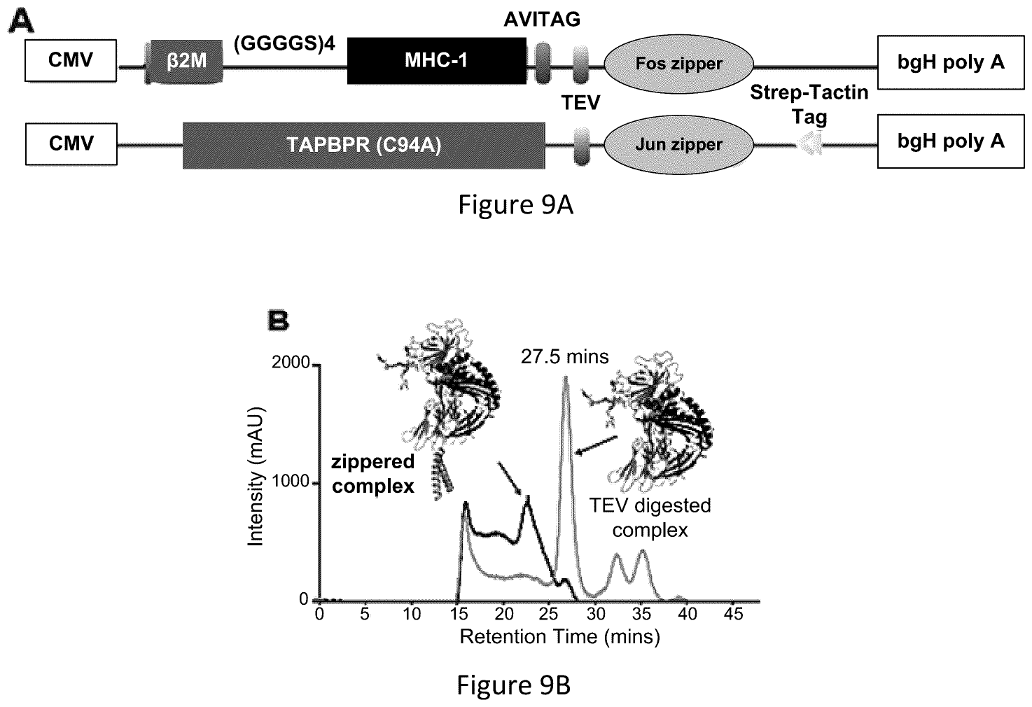

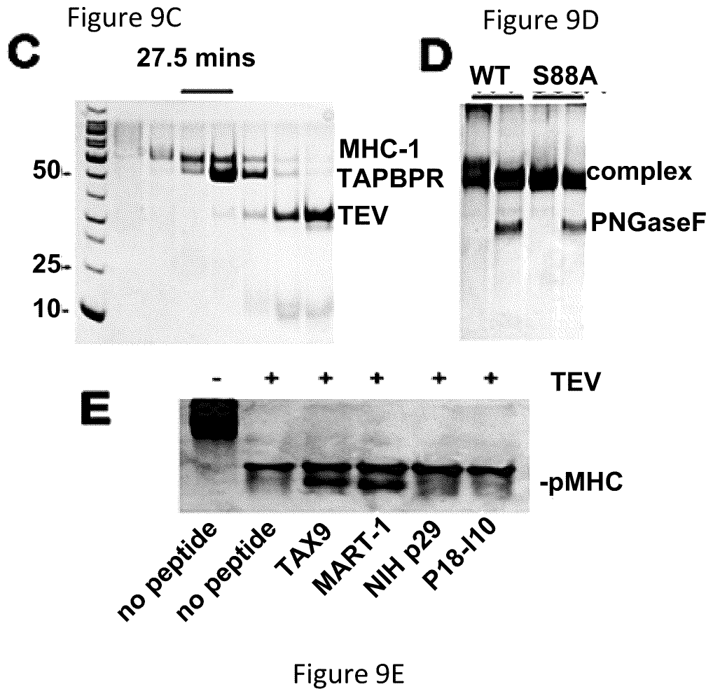

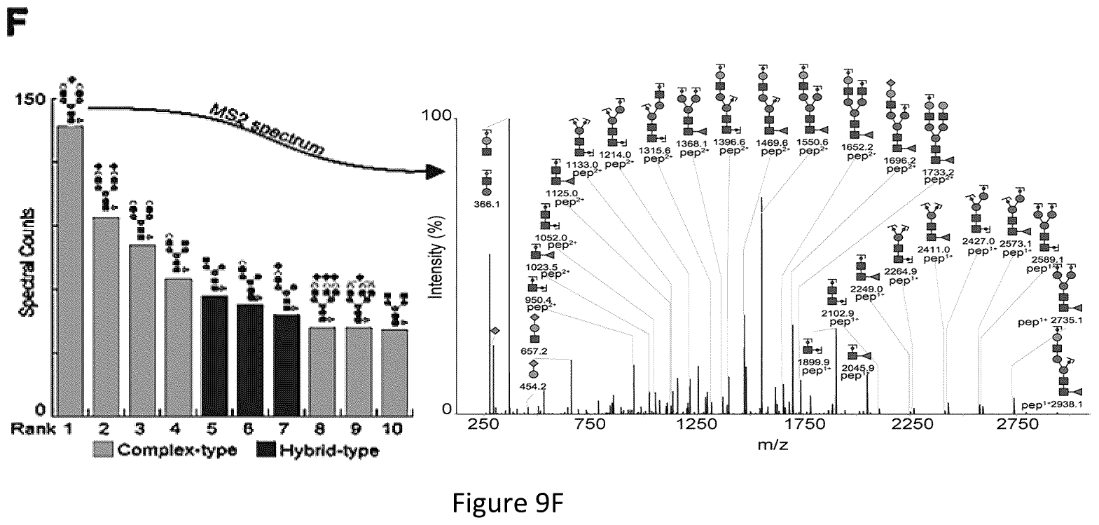

The full arrangement of the MHC-I and TAPBPR transgenes engineered to co-express a leucine zippered MHC-I/TAPBPR complex is shown in FIG. 9A. First, a CMV expression cassette was constructed that incorporates the endogenous human secretory β2m signal-peptide and coding sequences linked via a (GGGS)₄ (SEQ ID NO:47) spacer to the ectodomain of human allele HLA-A*02:01 (Hansen et al., Trends Immunol., 31, 363-369 (2010)). The gene was further engineered to express an AviTag sequence for in vitro biotinylation (Fairhead and Howarth, Methods Mol. Biol. Clifton NJ, 1266, 171-184 (2015)), and a FOS leucine coil motif (Kouzarides and Ziff, Nature, 336, 646-651 (1988)). The TAPBPR expression cassette similarly comprises a secretory peptide, the TAPBPR ectodomain (with a single C94A mutation), a tag for affinity purification, and a JUN leucine-coil motif. All sequences included a Tobacco Etch Virus (TEV) protease site for leucine zipper removal. Co-expression of the zippered single-chain and TAPBPR proteins permitted folding of the β2m and heavy-chain domains to yield peptide receptive MHC-I complexes. The peptide receptive MHC-I complex was secreted and purified directly from conditioned media. As a proof of concept, pilot studies were small scale with no media optimization (100 mL volume resulting in 10-30 mg/L of zippered complex per batch) but the process is highly scalable (Steger et al., J. Biomol. Screen., 20, 545-551 (2015)). After TEV digestion, the complex remained stable during preparative chromatography (FIG. 9B), and the individual MHC-I and TAPBPR components were resolved using SDS-PAGE as discrete protein bands with apparent molecular mass 51 and 43.5 kDa, respectively (FIG. 9C). The CHO-derived peptide receptive MHC-I complex was fully glycosylated, as shown by PNGaseF cleavage of all N-linked oligosaccharides from the wildtype molecule resulting in a distinct band shift, but not from the S88A MHC-I mutant (lacking the N-X-S/T glycosylation motif (Yan and Lennarz, J. Biol. Chem., 280, 3121-3124 (2005)) (FIG. 9D).

An electrophoretic mobility shift assay on a non-denaturing (native) gel was used to further confirm that the chaperoned MHC-I protein was peptide-receptive (Morozov et al., Proc. Natl. Acad. Sci. U.S.A., 113, E1006-E1015 (2016)). In this assay, incubation of the cleaved complex with 10-fold molar excess of two high-affinity peptides (TAX9 or MART-1), led to the formation of discrete bands corresponding to properly conformed pMHC species of slightly different electrophoretic mobilities due to the charge of bound peptides (FIG. 9E, lanes 3 and 4). In contrast, incubation with buffer or two non-specific peptides produced no distinguishable pMHC band (FIG. 9E, lanes 2, 5 and 6), while the leucine-zippered complex barely entered the gel (FIG. 9E, lane 1). Following protein purification using preparative size exclusion chromatography, the cleaved complex was snap frozen and stored at −80° C. for up to several months while remaining peptide-receptive.

### Complex-Type Glycosylation Patterns of MHC-I Molecules

Glycans play important roles in the immune response by affecting folding, multimerization, trafficking, cell surface stability and half-life of both antigens and their receptors (Baum and Cobb, Glycobiology, 27, 619-624 (2017)). Despite the highly polymorphic nature of HLA-A, HLA-B, and HLA-C alleles, all class-I molecules share a conserved glycan at Asn 86, and the oligosaccharide structures that predominate appear to be highly processed, biantennary N-linked-oligosaccharides (Parham et al., J. Biol. Chem., 252, 7555-7567 (1977)). Mass-spectroscopy confirmed that proteolysis of CHO-derived recombinant MHC-I resulted in isolation of a single N-glycosylation site at N86 within a unique 15-residue fragment. Peaks corresponding to this peptide revealed high intensities in both the MS1 and MS2 dimensions following analysis by ESI-MS/MS (FIG. 9F). One predominant peak in the MS2 spectra of all N86 glycopeptide species was observed, corresponded to peptide GYYNQSEAGSHTVQR (SEQ ID NO:51) from MHC-I, plus a single N-acetylhexosamine residue in MS2 spectra. The first and second most abundant species of glycan detected were highly-processed, biantennary disialylated N86-glycans containing fucose and one or two terminal N-acetylneuraminic acid residues, respectively. This result is similar to those reported in previous studies using primary and immortalized human cells (Barber et al., J. Immunol., 156, 3275-3284 (1996)), suggesting that the CHO-expressed MHC-I molecules recapitulate functionally critical glycan modifications.

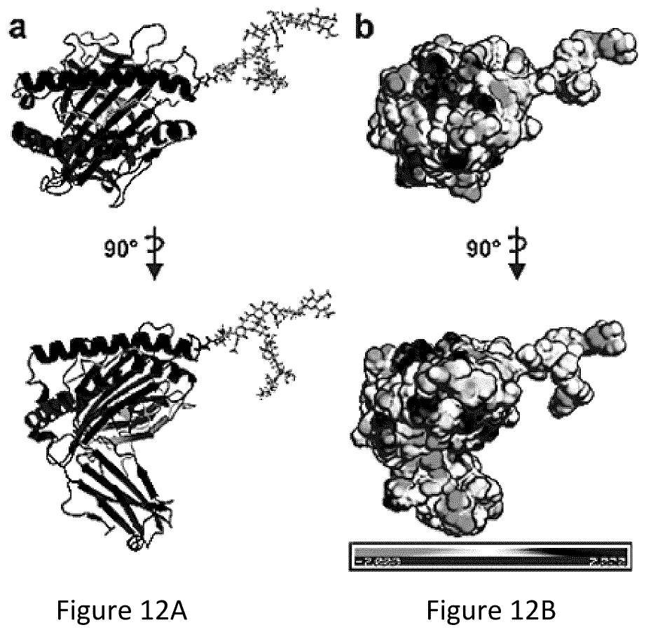

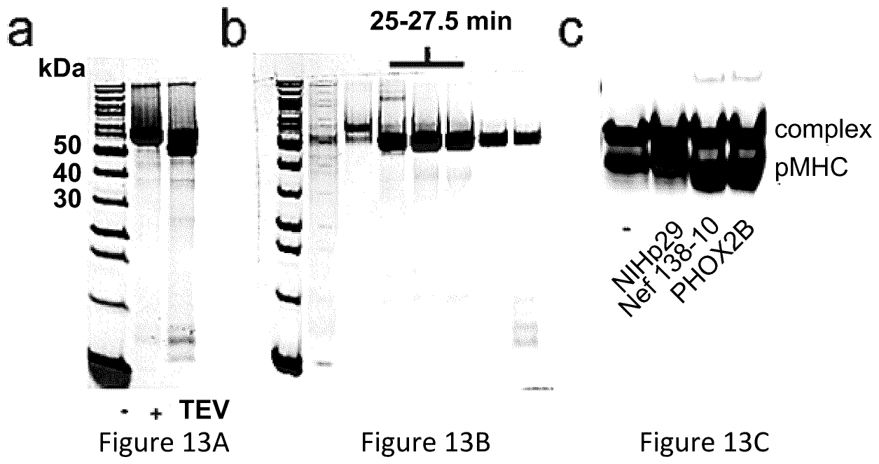

A biantennary glycan bearing two non-reducing terminal sialic acid residues on the HLA-A*02:01/MART-1 X-ray structure (FIG. 12A) (Sliz et al., J. Immunol., 167, 3276-3284 (2001)). The glycan moiety occupies a region adjacent to the F-pocket of the peptide binding groove, and overlaps with the Bw4 epitope recognized by the KIR3DL1 natural killer receptor, consistent with reported effects of glycan modifications on NK cell function (Salzberger et al., PLOS ONE, 10, e0145324 (2015)). Finally, potential long-range effects of the charged glycan on pMHC-I surface chemistry were considered, by calculating the electrostatic potential using CHARMM (Jo et al., J. Comput. Chem., 29, 1859-1865 (2008)). Display of the potential features on the X-ray structure of HLA-A*02:01 reveals a significant effect on the characteristics of the molecular surface that is displayed to T cell receptors, suggesting the utility of glycosylated pMHC-I proteins as molecular probes of functionally relevant interactions with T cells (FIG. 12B).

### Screening Peptide Binding on CHO-Derived MHC-I/TAPBPR Complexes

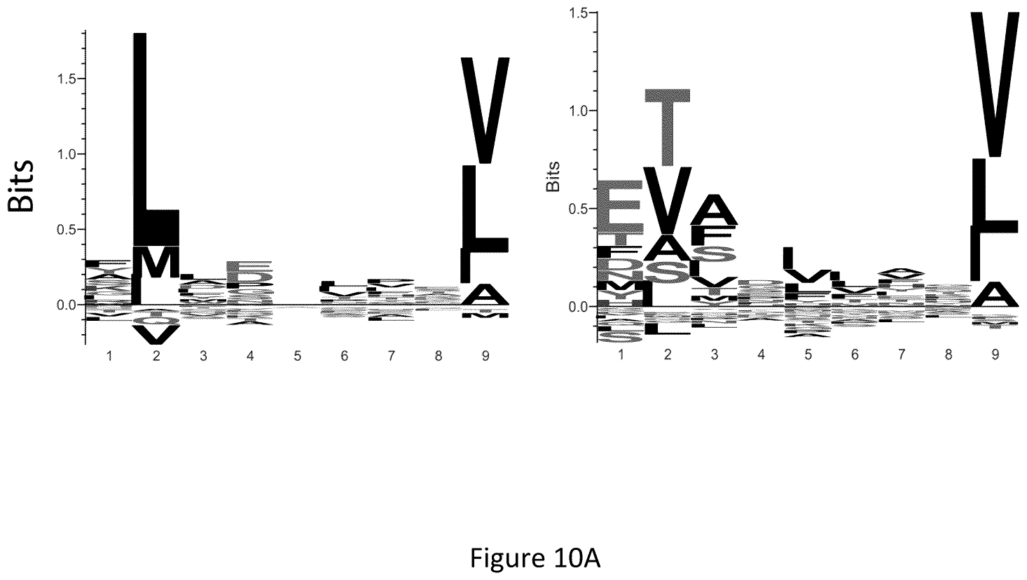

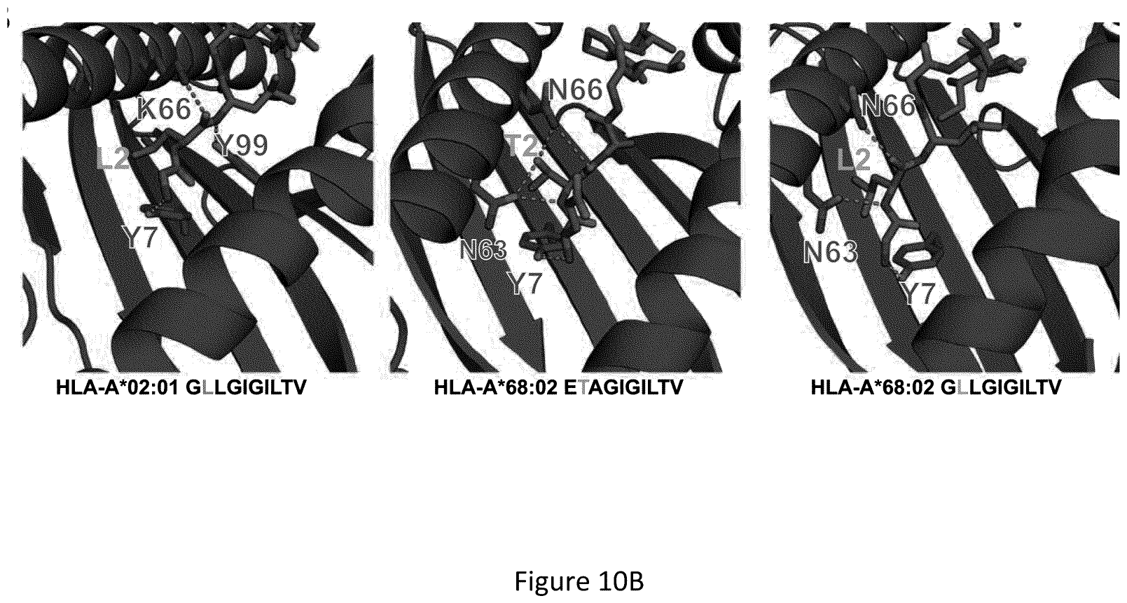

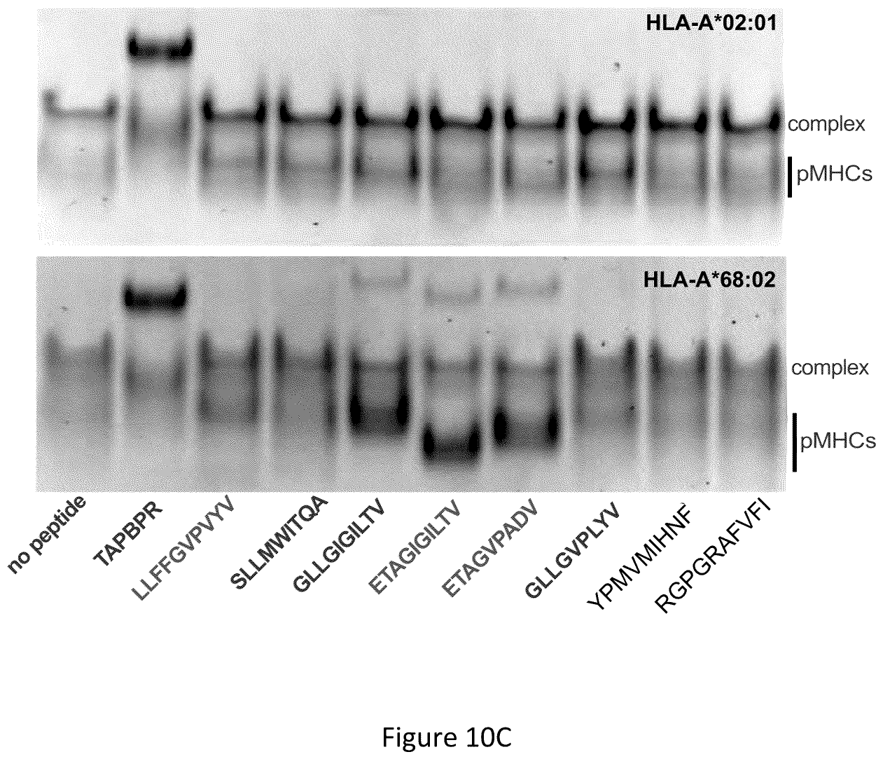

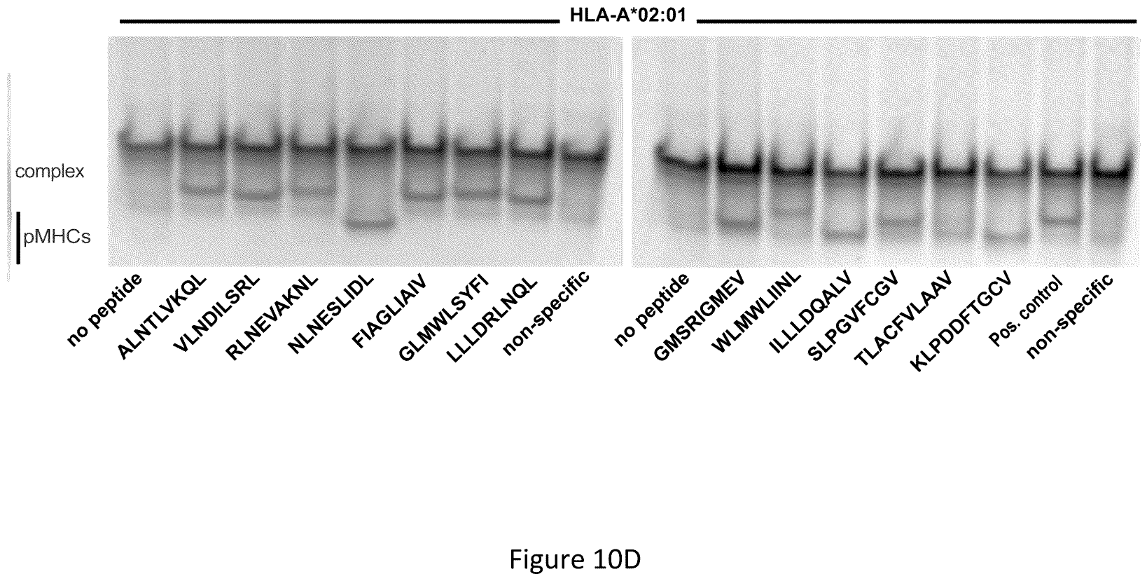

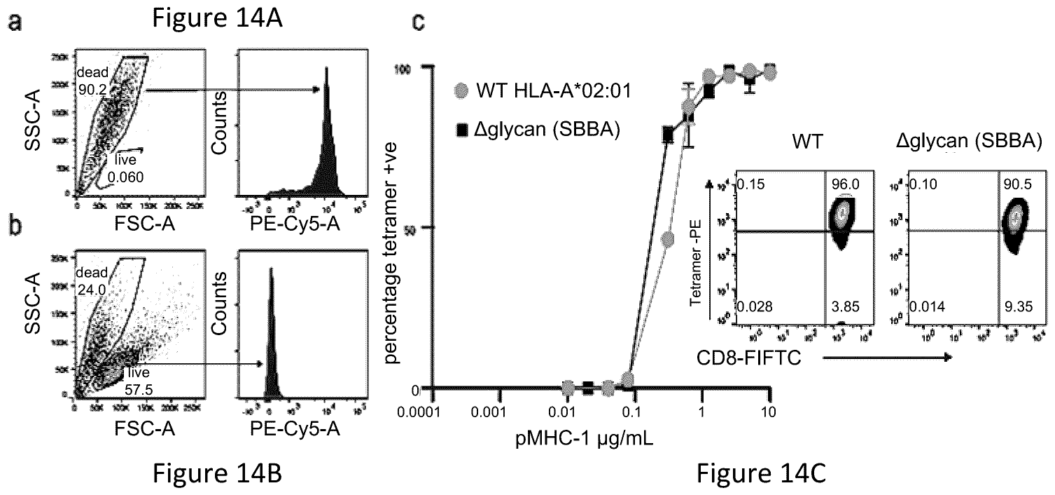

CHO-derived HLA-A*02:01/TAPBPR complexes can be loaded with high-affinity TAX9 and MART-1 peptides (FIG. 10D). To test if other MHC-I allotypes might be assembled and isolated using our system, a single chain HLA-A*68:02/TAPBPR complex was made and assayed it for pMHC-I formation in parallel with HLA-A*02:01. The two alleles have considerable sequence homology in the peptide-binding groove formed by the α1/α2 domains, resulting in a shared preference for a Val, Leu, Ala or Ile at the peptide anchor position 9, but marked variation at anchor position 2 where HLA-A*02:01 has a preference for Leu while HLA-A*68:02 prefers Thr (FIG. 10A). Guided by NetMHCpan (Jurtz et al., J. Immunol. Baltim. Md 1950, 199, 3360-3368 (2017)), a peptide panel was selected that included high- to low-affinity binders for HLA-A*02:01 and HLA-A*68:02, and directly probed pMHC-I formation using the described system. Structure-based modeling (Nerli and Sgourakis, bioRxiv, 2020.03.23.004176 (2020)) confirmed A-pocket hydrogen bonding interactions between peptide anchor position L2 with the K66 sidechain, Y7 with Y99 from HLA-A*02:01, and anchor position T2 with N66 and N63 from HLA-A*68:02 (FIG. 10B). According to the electrophoretic mobility shift assay, all predicted high-affinity peptides bound to HLA-A*02:01 and HLA-A*68:02 resulting in discrete pMHC-I protein bands. (FIG. 10C). These results demonstrate that the HLA-A*68:02/TAPBPR complex is stable and peptide-receptive, and that differences in peptide binding preferences can be recapitulated using the described chaperone-based system suggesting that the isolated MHC-I/TAPBPR complexes exhibit a properly conformed peptide-binding groove. The single, unexpected result was significant binding of peptide GLLGIGILTV (SEQ ID NO:3) on HLA-A*68:02, despite the high μM predicted affinity by NetMHCpan (Jurtz et al., J. Immunol. Baltim. Md 1950, 199, 3360-3368 (2017)). Structural modelling of the GLLGIGILTV (SEQ ID NO:3)/HLA A*68:02 complex (FIG. 10B-right panel) suggested the potential for stabilizing interactions at both peptide anchor positions, with leucine (L2) capable of hydrogen-bonding with N63 and N66. These results were expanded to demonstrate peptide-receptive HLA-A*24:02/TAPBPR complex formation (FIG. 14), suggesting the broad utility of the disclosed system for producing native pMHC-I complexes across allotypes of the HLA-A02 supertype (Ilca et al., Cell Rep., 29, 1621-1632.e3 (2019); (Overall et al., Nat. Commun., 11, 1-13 (2020)).

Peptide/MHC-I complex assembly for a panel of HLA-A*02:01-restricted epitopic peptides predicted from the SARS-COV-2 genome was demonstrated using a developed structure-guided method developed (Nerli and Sgourakis, bioRxiv, 2020.03.23.004176 (2020)) (Table 1). Peptides were incubated with MHC-I/TAPBPR complex, then analyzed for binding using the described electrophoretic mobility shift assay. Of 13 predicted peptides, 11 produced strong pMHC-I bands. The two peptides that were predicted to be weak binders (TLACFVLAAV (SEQ ID NO: 23) and WLMWLIINL (SEQ ID NO:20)) had little or no binding, as evidenced by no pMHC-I band formation. The specificity of peptide binding is reflected by the observed electrophoretic mobilities of the resulting pMHC-I species, which correlate with the overall charges and hydrodynamic radii of the resulting protein complexes (FIG. 10D).

TABLE 1

| | | | netMHCpan | netMHCpan | netMHCpan |
|---|---|---|---|---|---|
| | | | predicted | predicted | predicted |
| | | | affinity (nM) | affinity (nM) | affinity (nM) |
| Peptide | Sequence | Length | HLA:A*02:01 | HLA:A*68:02 | HLA:A*24:02 |
| TAX9 | LLFGVPVYV | 9 | 3.4 | 64 | — |
| | SEQ ID NO: 1 | | | | |
| NY-ESO-1 | SLLMWITQA | 9 | 23 | 7297 | — |
| | SEQ ID NO: 2 | | | | |
| GM1 | GLLGIGILTV | 10 | 15.9 | 12160.2 | — |
| | SEQ ID NO: 3 | | | | |
| GM2 | ETAGVPADV | 9 | 14820.3 | 5.3 | — |
| | SEQ ID NO: 4 | | | | |
| GM3 | ETAGIGILTV | 10 | 4436.9 | 4.4 | — |
| | (SEQ ID NO: 5) | | | | |
| GM4 | GLLGVPLYV | 9 | 5.4 | 4662.9 | — |
| | (SEQ ID NO: 6) | | | | |
| NIH p29 | YPNVNIHNF | 9 | 22457.6 | 13418.9 | — |
| | (SEQ ID NO: 7) | | | | |
| P18_I10 | RGPGRAFVFI | 10 | 17132.2 | 12829.9 | — |
| | (SEQ ID NO: 8) | | | | |
| MART-1 | EAAGIGILTV | 10 | 5322.6 | — | |
| | (SEQ ID NO: 9) | | | | |
| PHOX2B | VYGFVRACL | 9 | — | — | 466.54 |
| | (SEQ ID NO: 10) | | | | |
| Nef138-10 | RYPLTFGWCF | 10 | — | — | 6.57 |
| | (SEQ ID NO: 11) | | | | |

Sars-CoV-2 HLA-A02:01 restricted peptides.

| | | | netMHCpan | | |
|---|---|---|---|---|---|
| Swiss-Model | | | predicted | Structure- | |
| Repository | | | affinity (nM) | Based | |
| Reference | Sequence | Length | HLA:A*02:01 | model | charge |
| YP_009724390.1 | ALNTLVKQL | 9 | 1633.23 | Weak binder | +1 |
| | (SEQ ID NO: 12) | | | | |
| YP_009724390.1 | VLNDILSRL | 9 | 33.57 | Strong binder | 0 |
| | (SEQ ID NO: 13) | | | | |
| YP_009724390.1 | RLNEVAKNL | 9 | 940.92 | Strong binder | +1 |
| | (SEQ ID NO: 14) | | | | |
| YP_009724390.1 | NLNESLIDL | 9 | 177.32 | Strong binder | −2 |
| | (SEQ ID NO: 15) | | | | |
| YP_009724390.1 | FIAGLIAIV | 9 | 10.29 | Strong binder | 0 |
| | (SEQ ID NO: 16) | | | | |
| YP_009724392.1 | GLMWLSYFI | 9 | 3.87 | Strong binder | 0 |
| | (SEQ ID NO: 17) | | | | |
| YP_009724392.1 | LLLDRLNQL | 9 | 14.81 | Strong binder | 0 |
| | (SEQ ID NO: 18) | | | | |
| YP_009724389.1 | GMSRIGMEV | 9 | 50.61 | Strong binder | 0 |
| | (SEQ ID NO: 19) | | | | |
| YP_009724389.1 | WLMWLIINL | 9 | 6.60 | Weak binder | 0 |
| | (SEQ ID NO: 20) | | | | |
| YP_009724389.1 | ILLLDQALV | 9 | 27.11 | Strong binder | 0 |
| | (SEQ ID NO: 21) | | | | |
| YP_009724389.1 | SLPGVFCGV | 9 | 24.07 | Strong binder | −1 |
| | (SEQ ID NO: 22) | | | | |
| YP_009724393.1 | TLACFVLAAV | 10 | 20.28 | Strong binder | 0 |
| | (SEQ ID NO: 23) | | | | |
| YP_009724390.1 | KLPDDFTGCV | 10 | 77.11 | Strong binder | −1 |
| | (SEQ ID NO: 24) | | | | |

### Antigen-Specific T Cell Recognition of Native Peptide/HLA Antigens

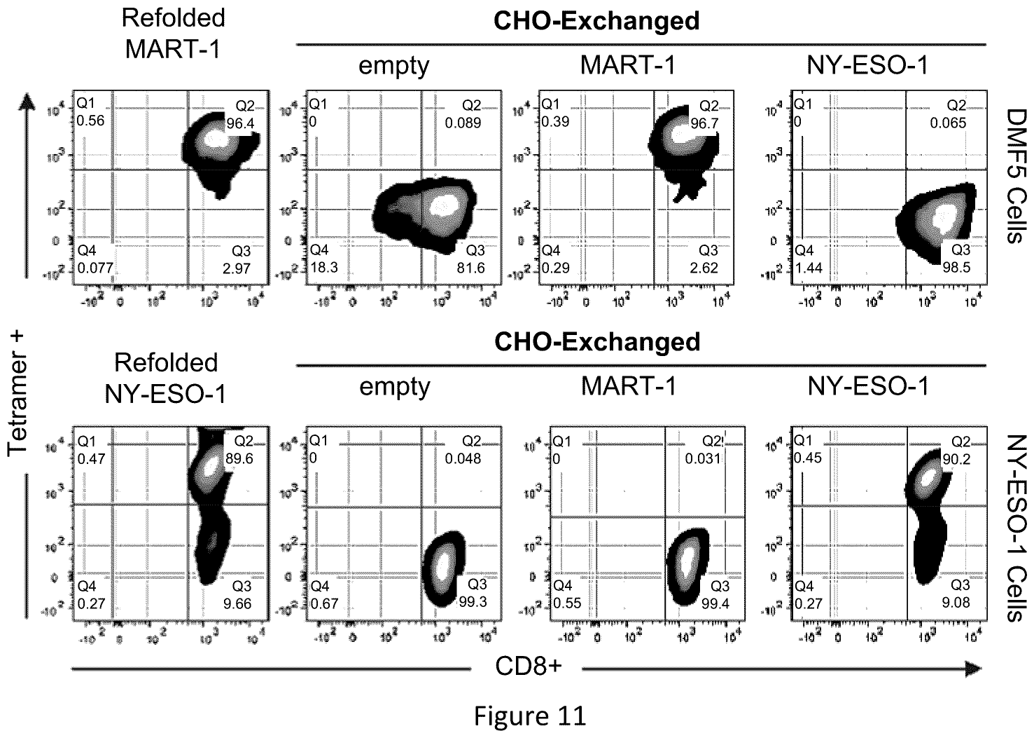

CHO-derived MHC-I/TAPBPR complexes may be readily multimerized via a streptavidin fluorophore conjugate (Altman et al., Science, 274, 94-96 (1996)). TAPBPR-promoted peptide loading can be then utilized to generate pMHC-I tetramers of desired peptide specificities. The efficiency of antigen-specific staining of a human lymphocyte line (DMF5) transduced with a T cell receptor (Melan-A) specific for the melanoma-associated MART-1 peptide (Johnson et al., J. Immunol. Baltim. Md 1950, 177, 6548-6559 (2006)) was demonstrated. DMF5 cells were incubated with phycoerythrin (PE) labelled MART-1/MHC-I tetramers prepared using either i) in vitro refolded pMHC-I (used as a positive control) vs ii) empty MHC/TAPBPR complexes or iii) TAPBPR-exchanged pMHC-I complexes loaded with the heteroclitic MART-1 peptide or iv) a different antigenic peptide, NY-ESO-1, corresponding to the cancer-testis antigen 1B (Gnjatic et al., Adv. Cancer Res., 95, 1-30 (2006)). To demonstrate antigen/receptor-specific tetramer staining, a complementary set of flow cytometry experiments was performed using a NY-ESO-1 specific T cell line (Bethune et al., Proc. Natl. Acad. Sci. U.S.A., 115, E10702-E10711 (2018)). Flow cytometry (FIGS. 11 and 14) showed that CHO-derived pMHC-I tetramers behaved in a manner comparable to tetramers generated from refolded proteins, specifically staining cells expressing the cognate but not the irrelevant T cell receptors, for both peptides.

### Discussion

Murine MHC-I molecules engineered as single-chain constructs with a covalently linked peptide were first expressed in mammalian cells (Mage et al., Proc. Natl. Acad. Sci. U.S.A., 89, 10658-10662 (1992)) and are reported to stimulate both antigen-specific B and T cells (Yu et al., 2002). Mammalian expression systems for human HLA antigens with the potential for immune stimulation include both single-chain constructs (Jurewicz et al., Anal. Biochem., 584, 113328 (2019)), and pMHC-IgG fusions (Wooster et al., J. Immunol. Methods, 464, 22-30 (2019)), but significant challenges remain with respect to loading such molecules with high-affinity peptides of choice. Here, systems and methods are provided to produce soluble, peptide-receptive human MHC-I proteins in the biopharmaceutical standard Chinse Hamster Ovary line, suitable for generating natively-folded and glycosylated pMHC-I with controlled peptide specificities. The above results demonstrate reconstitution of three commonly occurring human HLA allotypes of the A02 supertype (HLA-A*02:01, HLA-A*68:02 and HLA-A*24:02) (Sidney et al., BMC Immunol., 9, 1 (2008)) as fully functional, properly conformed pMHC-I complexes. The antigen specific staining of T cells with CHO-derived pMHC-I tetramers encompassing known tumor antigens is comparable to that of refolded pMHC-I complexes, while allowing for a significantly more convenient process which includes functionally important post translational modifications. Finally, an application of this platform was demonstrated to assay the specificity of several predicted epitopic peptides from the SARS-COV-2 genome against the common allotype HLA-A*02:01, which provides a convenient approach to validate peptide/HLA binding. Combined with previously described multiplexed tetramer (Bentzen and Hadrup, Annu. Rev. Immunol., 31, 443-473 (2017); Overall et al., Nat. Commun., 11, 1-13 (2020)) and nanoparticle methods (Ichikawa et al., Clin. Cancer Res. Off. J. Am. Assoc. Cancer Res., doi: 10.1158/1078-0432. CCR-19-3487 (2020)), the disclosed system can be leveraged for the development of antigen libraries to monitor and expand polyclonal T cell specificities in various research and clinical settings.

### Materials and Methods

Purification of peptide receptive MHC-I Complexes. Culture media was harvested and pre-cleared by centrifugation at 250×g for 10 minutes before adjusting to 25 mM Tris pH 8, 1 mM EDTA and adding 27 mg/L of avidin. The media was filtered (0.22 μm) and affinity purified on a StrepTrap HP affinity column (GE Healthcare, Chicago IL). Bound protein was washed with 10 column volumes of wash buffer (25 mM Tris pH 8, 100 mM NaCl, 1 mM EDTA) and eluted with 5 mM desthiobiotin/wash buffer. Leucine zippers were removed by a 2-hour digestion at room temperature with Tobacco Etch Protein (TEV) in 25 mM Tris pH 8, 100 mM NaCl, 1 mM EDTA, 3 mM/0.3 mM glutathione redox buffer. Complex was polished by size exclusion gel filtration (SEC) at room temperature on a Superdex 200 10/300 increase column (GE Healthcare, Chicago IL) at a flow rate of 0.5 mL/min in 50 mM Tris pH 7.5 buffer containing 100 mM NaCl. Protein concentrations were determined using A280 measurements on a NanoDrop spectrophotometer.

Native gel-shift assay of peptide binding peptide receptive MHC-I. Peptide-receptive MHC-I complexes were incubated with a 10-molar excess of high affinity or non-binding peptide overnight at 4° C. temperature at pH 7.2 in phosphate buffer with 150 mM NaCl. Samples were electrophoresed at 90V on a 12% polyacrylamide gel in 25 mM Tris pH 8.8, 192 mM glycine, at 4° C. for 5 hours, and developed using InstantBlue (Expedeon San Diego, CA). Gels were imaged using an Innotech FluoChem2 system (Genetic Technologies Grover, MO).

PNGase F digestion assay. Five μg aliquots of purified HLA-A*02:01 and HLA-A*02:01 S88A (ΔN86 glycan) TAPBPR complex were denatured then either treated with PNGase F (NEB, Ipswich, MA) or incubated in glycosidase buffer alone at 37° C. for 1 hour, then reduced with DTT and electrophoresed.

Glycan Mapping by LC-MS MS N-Glycan analysis. All materials were purchased from Millipore-Sigma unless otherwise noted. Purified MHC-I (20 μg) was buffer exchanged into 50 mM ammonium bicarbonate (pH 8.0) and incubated at 90° C. for 5 min. Following trypsin digestion and reduction, the sample was iodoacetamide treated. Glycopeptides were then enriched using the ProteoExtract Glycopeptide Enrichment Kit according to the manufacturing guidelines, and lyophilized before resuspension in 20 μL of 5% Acetonitrile and 0.1% Trifluoroacetic acid in ddH₂O. Glycopeptides (5 μL) were injected on to a 75 mm×20 cm column packed with C18 Zorbax resin equipped using a Thermo Scientific EASY-nLC 1200 nanopump. Analytes were eluted with a linear gradient of increasing acetonitrile and injected into a Q Exactive hybrid quadrupole mass spectrometer (Thermo Scientific). MS2 spectra for intense ions were collected with stepped NCE energies of 15, 25 and 35 eV. The 10 most abundant N-glycopeptides, based on spectra counts, were annotated using GlycoWorkbench Version 2.1

E. coli Protein expression, refolding and purification of conventional refolded pMHC-I. Luminal domain HLA-A*02:01 and human β2m expression plasmids were provided by the NIH Tetramer Core Facility. Proteins were expressed previously described and in vitro refolded in the presence of 10-fold molar excess of synthetic peptides.

Tetramer formation. MHC-I molecules were biotinylated using biotin ligase (BirA) (Avidity.com, Co.). Tetramerization of peptide receptive-MHC-I complexes was performed by adding a 2:1 molar ratio of biotinylated peptide receptive MHC-I to streptavidin-PE or streptavidin-APC (Prozyme Hayward, CA) in five additions over 1 hr on ice. Peptide-receptive MHC-Itetramers were then exchanged with peptides of interest by adding a 20-molar excess of peptide and incubating for 1 hour. A solution of 8M biotin (to block any free streptavidin sites) was added and incubated for a further 1 hr at room temperature. After exchange, tetramers were transferred to 100 kDa spin columns (Amicon, Millipore, Burlington, Ma) and washed with 1000 volumes of PBS to remove TAPBPR and excess peptide. For T-cell receptor staining comparison, conventionally produced MHC-I molecules were biotinylated and assembled onto streptavidin-PE.

Flow cytometry. Tetramer analysis of cell lines was carried out by staining 2×10⁵ DMF5 cells, with an anti-CD8α FITC conjugated mAb (BD Biosciences) and 1 μg/mL of either conventionally refolded PE-HLA-A*02:01/MART-1 tetramer, CHO-derived PE-HLA-A*02:01 tetramer loaded with MART-1 peptide, CHO-derived PE-HLA-A*02:01 tetramer loaded with the neo-antigen NY-ESO-1, or left peptide-free and incubated for 1 hr on ice. Cells were washed twice with 30 volumes of FACS buffer (PBS, 1% BSA, 2 mM EDTA) before analysis, and gated by forward and side scattering properties. All experiments were performed using an LSRII (BD) and data analysis performed using FACSDiva (BD) and FlowJo (Tree Star, Ashland Oregon). Cells tested negative for mycoplasma using the universal mycoplasma test kit (ATCC).

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

1-61. (canceled)

62. A heterodimeric MHC Class I (MHC-I)/Tapasin Binding Protein Related (TAPBPR) protein complex comprising
(a) a first polypeptide comprising, from N-terminus to C-terminus:
i) a MHC Class I heavy chain that is glycosylated on at least one native glycosylation position,
ii) a β2 microglobulin,
iii) a first protease cleavage site, and
iv) a first heterodimerization domain; and
(b) a second polypeptide comprising, from N-terminus to C-terminus:
i) a TAPBPR;
ii) a second protease cleavage site, and
iii) a second heterodimerization domain.

63. The heterodimeric MHC-I/TAPBPR protein complex of claim 62, wherein the first polypeptide comprises a multimerization tag.

64. The heterodimeric MHC-I/TAPBPR protein complex of claim 62, wherein the first polypeptide and second polypeptide each further comprise one or more purification tags.

65. The heterodimeric MHC-I/TAPBPR protein complex of claim 62, where the MHC Class I heavy chain is a human HLA-A, HLA-B, or HLA-C or a mouse H-2D or H-2L.

66. The heterodimeric MHC-I/TAPBPR protein complex of claim 65, where the MHC Class I heavy chain is an HLA-A*02:01, HLA-A*24:02, HLA-A*68:01 or HLA-A*68:02 allele heavy chain.

67. The heterodimeric MHC-I/TAPBPR protein complex of claim 62, where the MHC Class I heavy chain has one or more mutations in the a3 domain of the heavy chain.

68. The heterodimeric MHC-I/TAPBPR protein complex of claim 62, where the first and second protease cleavage sites are each a TEV protease cleavage site.

69. The heterodimeric MHC-I/TAPBPR protein complex of claim 63, where the multimerization tag is an AviTag.

70. The heterodimeric MHC-I/TAPBPR protein complex of claim 69, where the AviTag comprises a biotinylated lysine.

71. The heterodimeric MHC-I/TAPBPR protein complex, where the MHC Class I heavy chain is glycosylated at residue N86.

72. The heterodimeric MHC-I/TAPBPR protein complex of claim 62, where the first heterodimerization domain and second heterodimerization domain are each a leucine zipper domain.

73. An expression vector composition comprising:
a) a first polynucleotide encoding a first polypeptide, the first polypeptide comprising from N-terminus to C-terminus:
i) a β2 microglobulin,
ii) an MHC Class I (MHC-I) heavy chain that is glycosylated at at least one native glycosylation position,
iii) a first protease cleavage site, and
iv) a first heterodimerization domain; and
b) a second polynucleotide encoding a second polypeptide, the second polypeptide comprising from N-terminus to C-terminus:
i) a TAPBPR;
ii) a second protease cleavage site, and
iii) a second heterodimerization domain.

74. A mammalian host cell comprising the expression vector composition of claim 73.

75. The mammalian host cell of claim 74, where the cell line is a CHO or HEK cell line.

76. The mammalian cell line of claim 74, where the cell line is a CHO-K1 cell line.

77. A method of making a heterodimeric MHC Class I (MHC-I)/Tapasin Binding Protein Related (TAPBPR) protein complex comprising:
a) culturing the mammalian host cell of claim 3 under conditions wherein the heterodimeric MHC-I/TAPBPR protein complex is expressed; and
b) recovering the heterodimeric MHC-I/TAPBPR protein complex.
